SEQUENCE LISTING (24 sequences)

SEQ ID NO: 1 - DNA, 3309 bp, Homo sapiens
atgaccgcctgcgcccgccgagcgggtgggcttccggaccccgggctctgcggtcccgcgtggtgggctccgtccctgccccgcctcccccgggccctgccccggctcccgctcctgctgctcctgcttctgctgcagccccccgccctctccgccgtgttcacggtgggggtcctgggcccctgggcttgcgaccccatcttctctcgggctcgcccggacctggccgcccgcctggccgccgcccgcctgaaccgcgaccccggcctggcaggcggtccccgcttcgaggtagcgctgctgcccgagccttgccggacgccgggctcgctgggggccgtgtcctccgcgctggcccgcgtgtcgggcctcgtgggtccggtgaaccctgcggcctgccggccagccgagctgctcgccgaagaagccgggatcgcgctggtgccctggggctgcccctggacgcaggcggagggcaccacggcccctgccgtgacccccgccgcggatgccctctacgccctgcttcgcgcattcggctgggcgcgcgtggccctggtcaccgccccccaggacctgtgggtggaggcgggacgctcactgtccacggcactcagggcccggggcctgcctgtcgcctccgtgacttccatggagcccttggacctgtctggagcccgggaggccctgaggaaggttcgggacgggcccagggtcacagcagtgatcatggtgatgcactcggtgctgctgggtggcgaggagcagcgctacctcctggaggccgcagaggagctgggcctgaccgatggctccctggtcttcctgcccttcgacacgatccactacgccttgtccccaggcccggaggccttggccgcactcgccaacagctcccagcttcgcagggcccacgatgccgtgctcaccctcacgcgccactgtccctctgaaggcagcgtgctggacagcctgcgcagggctcaagagcgccgcgagctgccctctgacctcaatctgcagcaggtctccccactctttggcaccatctatgacgcggtcttcttgctggcaaggggcgtggcagaagcgcgggctgccgcaggtggcagatgggtgtccggagcagctgtggcccgccacatccgggatgcgcaggtccctggcttctgcggggacctaggaggagacgaggagcccccattcgtgctgctagacacggacgcggcgggagaccggctttttgccacatacatgctggatcctgcccggggctccttcctctccgccggtacccggatgcacttcccgcgtgggggatcagcacccggacctgacccctcgtgctggttcgatccaaacaacatctgcggtggaggactggagccgggcctcgtctttcttggcttcctcctggtggttgggatggggctggctggggccttcctggcccattatgtgaggcaccggctacttcacatgcaaatggtctccggccccaacaagatcatcctgaccgtggacgacatcacctttctccacccacatgggggcacctctcgaaaggtggcccaggggagtcgatcaagtctgggtgcccgcagcatgtcagacattcgcagcggccccagccaacacttggacagccccaacattggtgtctatgagggagacagggtttggctgaagaaattcccaggggatcagcacatagctatccgcccagcaaccaagacggccttctccaagctccaggagctccggcatgagaacgtggccctctacctggggcttttcctggctcggggagcagaaggccctgcggccctctgggagggcaacctggctgtggtctcagagcactgcacgcggggctctcttcaggacctcctcgctcagagagaaataaagctggactggatgttcaagtcctccctcctgctggaccttatcaagggaataaggtatctgcaccatcgaggcgtggctcatgggcggctgaagtcacggaactgcatagtggatggcagattcgtactcaagatcactgaccacggccacgggagactgctggaagcacagaaggtgctaccggagcctcccagagcggaggaccagctgtggacagccccggagctgcttagggacccagccctggagcgccggggaacgctggccggcgacgtctttagcttggccatcatcatgcaagaagtagtgtgccgcagtgccccttatgccatgctggagctcactcccgaggaagtggtgcagagggtgcggagcccccctccactgtgtcggcccttggtgtccatggaccaggcacctgtcgagtgtatcctcctgatgaagcagtgctgggcagagcagccggaacttcggccctccatggaccacaccttcgacctgttcaagaacatcaacaagggccggaagacgaacatcattgactcgatgcttcggatgctggagcagtactctagtaacctggaggatctgatccgggagcgcacggaggagctggagctggaaaagcagaagacagaccggctgcttacacagatgctgcctccgtctgtggctgaggccttgaagacggggacaccagtggagcccgagtactttgagcaagtgacactgtactttagtgacattgtgggcttcaccaccatctctgccatgagtgagcccattgaggttgtggacctgctcaacgatctctacacactctttgatgccatcattggttcccacgatgtctacaaggtggagacaataggggacgcctatatggtggcctcggggctgccccagcggaatgggcagcgacacgcggcagagatcgccaacatgtcactggacatcctcagtgccgtgggcactttccgcatgcgccatatgcctgaggttcccgtgcgcatccgcataggcctgcactcgggtccatgcgtggcaggcgtggtgggcctcaccatgccgcggtactgcctgtttggggacacggtcaacaccgcctcgcgcatggagtccaccgggctgccttaccgcatccacgtgaacttgagcactgtggggattctccgtgctctggactcgggctaccaggtggagctgcgaggccgcacggagctgaagggcaagggcgccgaggacactttctggctagtgggcagacgcggcttcaacaagcccatccccaaaccgcctgacctgcaaccggggtccagcaaccacggcatcagcctgcaggagatcccacccgagcggcgacggaagctggagaaggcgcggccgggccagttctct

SEQ ID NO: 2 - protein, 1103 aa, Homo sapiens
MTACARRAGGLPDPGLCGPAWWAPSLPRLPRALPRLPLLLLLLLLQPPALSAVFTVGVLGPWACDPIFSRARPDLAARLAAARLNRDPGLAGGPRFEVALLPEPCRTPGSLGAVSSALARVSGLVGPVNPAACRPAELLAEEAGIALVPWGCPWTQAEGTTAPAVTPAADALYALLRAFGWARVALVTAPQDLWVEAGRSLSTALRARGLPVASVTSMEPLDLSGAREALRKVRDGPRVTAVIMVMHSVLLGGEEQRYLLEAAEELGLTDGSLVFLPFDTIHYALSPGPEALAALANSSQLRRAHDAVLTLTRHCPSEGSVLDSLRRAQERRELPSDLNLQQVSPLFGTIYDAVFLLARGVAEARAAAGGRWVSGAAVARHIRDAQVPGFCGDLGGDEEPPFVLLDTDAAGDRLFATYMLDPARGSFLSAGTRMHFPRGGSAPGPDPSCWFDPNNICGGGLEPGLVFLGFLLVVGMGLAGAFLAHYVRHRLLHMQMVSGPNKIILTVDDITFLHPHGGTSRKVAQGSRSSLGARSMSDIRSGPSQHLDSPNIGVYEGDRVWLKKFPGDQHIAIRPATKTAFSKLQELRHENVALYLGLFLARGAEGPAALWEGNLAVVSEHCTRGSLQDLLAQREIKLDWMFKSSLLLDLIKGIRYLHHRGVAHGRLKSRNCIVDGRFVLKITDHGHGRLLEAQKVLPEPPRAEDQLWTAPELLRDPALERRGTLAGDVFSLAIIMQEVVCRSAPYAMLELTPEEVVQRVRSPPPLCRPLVSMDQAPVECILLMKQCWAEQPELRPSMDHTFDLFKNINKGRKTNIIDSMLRMLEQYSSNLEDLIRERTEELELEKQKTDRLLTQMLPPSVAEALKTGTPVEPEYFEQVTLYFSDIVGFTTISAMSEPIEVVDLLNDLYTLFDAIIGSHDVYKVETIGDAYMVASGLPQRNGQRHAAEIANMSLDILSAVGTFRMRHMPEVPVRIRIGLHSGPCVAGVVGLTMPRYCLFGDTVNTASRMESTGLPYRIHVNLSTVGILRALDSGYQVELRGRTELKGKGAEDTFWLVGRRGFNKPIPKPPDLQPGSSNHGISLQEIPPERRRKLEKARPGQFS

SEQ ID NO: 3 - DNA, 3309 bp, Homo sapiens
atgaccgcctgcgcccgccgagcgggtgggcttccggaccccgggctctgcggtcccgcgtggtgggctccgtccctgccccgcctcccccgggccctgccccggctcccgctcctgctgctcctgcttctgctgcagccccccgccctctccgccgtgttcacggtgggggtcctgggcccctgggcttgcgaccccatcttctctcgggctcgcccggacctggccgcccgcctggccgccgcccgcctgaaccgcgaccccggcctggcaggcggtccccgcttcgaggtagcgctgctgcccgagccttgccggacgccgggctcgctgggggccgtgtcctccgcgctggcccgcgtgtcgggcctcgtgggtccggtgaaccctgcggcctgccggccagccgagctgctcgccgaagaagccgggatcgcgctggtgccctggggctgcccctggacgcaggcggagggcaccacggcccctgccgtgacccccgccgcggatgccctctacgccctgcttcgcgcattcggctgggcgcgcgtggccctggtcaccgccccccaggacctgtgggtggaggcgggacgctcactgtccacggcactcagggcccggggcctgcctgtcgcctccgtgacttccatggagcccttggacctgtctggagcccgggaggccctgaggaaggttcgggacgggcccagggtcacagcagtgatcatggtgatgcactcggtgctgctgggtggcgaggagcagcgctacctcctggaggccgcagaggagctgggcctgaccgatggctccctggtcttcctgcccttcgacacgatccactacgccttgtccccaggcccggaggccttggccgcactcgccaacagctcccagcttcgcagggcccacgatgccgtgctcaccctcacgcgccactgtccctctgaaggcagcgtgctggacagcctgcgcagggctcaagagcgccgcgagctgccctctgacctcaatctgcagcaggtctccccactctttggcaccatctatgacgcggtcttcttgctggcaaggggcgtggcagaagcgcgggctgccgcaggtggcagatgggtgtccggagcagctgtggcccgccacatccgggatgcgcaggtccctggcttctgcggggacctaggaggagacgaggagcccccattcgtgctgctagacacggacgcggcgggagaccggctttttgccacatacatgctggatcctgcccggggctccttcctctccgccggtacccggatgcacttcccgcgtgggggatcagcacccggacctgacccctcgtgctggttcgatccaaacaacatctgcggtggaggactggagccgggcctcgtctttcttggcttcctcctggtggttgggatggggctggctggggccttcctggcccattatgtgaggcaccggctacttcacatgcaaatggtctccggccccaacaagatcatcctgaccgtggacgacatcacctttctccacccacatgggggcacctctcgaaaggtggcccaggggagtcgatcaagtctgggtgcccgcagcatgtcagacattcgcagcggccccagccaacacttggacagccccaacattggtgtctatgagggagacagggtttggctgaagaaattcccaggggatcagcacatagctatccgcccagcaaccaagacggccttctccaagctccaggagctccggcatgagaacgtggccctctacctggggcttttcctggctcggggagcagaaggccctgcggccctctgggagggcaacctggctgtggtctcagagcactgcacgcggggctctcttcaggacctcctcgctcagagagaaataaagctggactggatgttcaagtcctccctcctgctggaccttatcaagggaataaggtatctgcaccatcgaggcgtggctcatgggcggctgaagtcacggaactgcatagtggatggcagattcgtactcaagatcactgaccacggccacgggagactgctggaagcacagaaggtgctaccggagcctcccagagcggaggaccagctgtggacagccccggagctgcttagggacccagccctggagcgccggggaacgctggccggcgacgtctttagcttggccatcatcatgcaagaagtagtgtgccgcagtgccccttatgccatgctggagctcactcccgaggaagtggtgcagagggtgcggagcccccctccactgtgtcggcccttggtgtccatggaccaggcacctgtcgagtgtatcctcctgatgaagcagtgctgggcagagcagccggaacttcggccctccatggaccacaccttcgacctgttcaagaacatcaacaagggccggaagacgaacatcattgactcgatgcttcggatgctggagcagtactctagtaacctggaggatctgatccgggacagcacggaggagctggagctggaaaagcagaagacagaccggctgcttacacagatgctgcctccgtctgtggctgaggccttgaagacggggacaccagtggagcccgagtactttgagcaagtgacactgtactttagtgacattgtgggcttcaccaccatctctgccatgagtgagcccattgaggttgtggacctgctcaacgatctctacacactctttgatgccatcattggttcccacgatgtctacaaggtggagacaataggggacgcctatatggtggcctcggggctgccccagcggaatgggcagcgacacgcggcagagatcgccaacatgtcactggacatcctcagtgccgtgggcactttccgcatgcgccatatgcctgaggttcccgtgcgcatccgcataggcctgcactcgggtccatgcgtggcaggcgtggtgggcctcaccatgccgcggtactgcctgtttggggacacggtcaacaccgcctcgcgcatggagtccaccgggctgccttaccgcatccacgtgaacttgagcactgtggggattctccgtgctctggactcgggctaccaggtggagctgcgaggccgcacggagctgaagggcaagggcgccgaggacactttctggctagtgggcagacgcggcttcaacaagcccatccccaaaccgcctgacctgcaaccggggtccagcaaccacggcatcagcctgcaggagatcccacccgagcggcgacggaagctggagaaggcgcggccgggccagttctct

SEQ ID NO: 4 - protein, 1103 aa, Homo sapiens
  MISC_FEATURE (837)..(837): Xaa=E or D
MTACARRAGGLPDPGLCGPAWWAPSLPRLPRALPRLPLLLLLLLLQPPALSAVFTVGVLGPWACDPIFSRARPDLAARLAAARLNRDPGLAGGPRFEVALLPEPCRTPGSLGAVSSALARVSGLVGPVNPAACRPAELLAEEAGIALVPWGCPWTQAEGTTAPAVTPAADALYALLRAFGWARVALVTAPQDLWVEAGRSLSTALRARGLPVASVTSMEPLDLSGAREALRKVRDGPRVTAVIMVMHSVLLGGEEQRYLLEAAEELGLTDGSLVFLPFDTIHYALSPGPEALAALANSSQLRRAHDAVLTLTRHCPSEGSVLDSLRRAQERRELPSDLNLQQVSPLFGTIYDAVFLLARGVAEARAAAGGRWVSGAAVARHIRDAQVPGFCGDLGGDEEPPFVLLDTDAAGDRLFATYMLDPARGSFLSAGTRMHFPRGGSAPGPDPSCWFDPNNICGGGLEPGLVFLGFLLVVGMGLAGAFLAHYVRHRLLHMQMVSGPNKIILTVDDITFLHPHGGTSRKVAQGSRSSLGARSMSDIRSGPSQHLDSPNIGVYEGDRVWLKKFPGDQHIAIRPATKTAFSKLQELRHENVALYLGLFLARGAEGPAALWEGNLAVVSEHCTRGSLQDLLAQREIKLDWMFKSSLLLDLIKGIRYLHHRGVAHGRLKSRNCIVDGRFVLKITDHGHGRLLEAQKVLPEPPRAEDQLWTAPELLRDPALERRGTLAGDVFSLAIIMQEVVCRSAPYAMLELTPEEVVQRVRSPPPLCRPLVSMDQAPVECILLMKQCWAEQPELRPSMDHTFDLFKNINKGRKTNIIDSMLRMLEQYSSNLEDLIRXXXEELELEKQKTDRLLTQMLPPSVAEALKTGTPVEPEYFEQVTLYFSDIVGFTTISAMSEPIEVVDLLNDLYTLFDAIIGSHDVYKVETIGDAYMVASGLPQRNGQRHAAEIANMSLDILSAVGTFRMRHMPEVPVRIRIGLHSGPCVAGVVGLTMPRYCLFGDTVNTASRMESTGLPYRIHVNLSTVGILRALDSGYQVELRGRTELKGKGAEDTFWLVGRRGFNKPIPKPPDLQPGSSNHGISLQEIPPERRRKLEKARPGQFS

SEQ ID NO: 5 - DNA, 244 bp, Sus scrofa
ctcccccatcccagcagcaagagcttgctaatcttttagctactaatcttttagccactaatctgatttccaaactgttggcacctgagctatttataaagcagtatttcatccccccagaagcctgttcttctcccctgacccccaccaatctaaaaactcagaggaccttgggtataagaggttgggcaggcgagcatagcaaccagagctggagacggatgtgagcttcatcttactcccc

SEQ ID NO: 6 - DNA, 725 bp, Sus scrofa
cttgttaccttcctctccaacaagttcaagtctcacagatgtgcacactcagctcaatacactcagcctcccctcccccatcccacccccattgacaggagattgactcctgctgtgcacataagctgggataatgggggggggcactttctaaacatcgcttcaacagtcccaagtcccgagtagtggggggctggggaaggggtgctctttcccatacccttggcttttgtgtggcctgtaatacctgatcaagagatatagaaagactggagtgtgacactaggctctcctttcagaatcagaaagtccaatgctttgagccctcctatttctatccttcaccttgcttttcttttaatgtccctgactcgcctttgatctctggccctcaggttcaaggcctcaaaaggccaaaaccctgtagttacccttctcaagctcctctgactttaacaccatcagattcaaccactgacccctcccccatcccagcagcaagagcttgctaatcttttagctactaatcttttagccactaatctgatttccaaactgttggcacctgagctatttataaagcagtatttcatccccccagaagcctgttcttctcccctgacccccaccaatctaaaaactcagaggaccttgggtataagaggttgggcaggcgagcatagcaaccagagctggagacggatgtgagcttcatcttactcccc

SEQ ID NO: 7 - DNA, 12667 bp, Artificial Sequence
  Lentiviral vector plasmid sequence Pt75 (short)
ttgtacaaagtggtgatgggtcaccatctagatggccatatgatcgatacgtactagtctcgacggtacctttaagaccaatgacttacaaggcagctgtagatcttagccactttttaaaagaaaaggggggactggaagggctaattcactcccaacgaagacaaaatcgtcgagagatgctgcatataagcagctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggccttgacattataatagatttagcaggaattgaactaggagtggagcacacaggcaaagctgcagaagtacttggaagaagccaccagagatactcacgattctgcacatacctggctaatcccagatcctaaggattacattaagtttactaacatttatataatgatttatagtttaaagtataaacttatctaatttactattctgacagatattaattaatcctcaaatatcataagagatgattactattatccccatttaacacaagaggaaactgagagggaaagatgttgaagtaattttcccacaattacagcatccgttagttacgactctatgatcttctgacacaaattccatttactcctcaccctatgactcagtcgaatatatcaaagttatggacattatgctaagtaacaaattacccttttatatagtaaatactgagtagattgagagaagaaattgtttgcaaacctgaatagcttcaagaagaagagaagtgaggataagaataacagttgtcatttaacaagttttaacaagtaacttggttagaaagggattcaaatgcataaagcaagggataaatttttctggcaacaagactatacaatataaccttaaatatgacttcaaataattgttggaacttgataaaactaattaaatattattgaagattatcaatattataaatgtaatttacttttaaaaagggaacatagaaatgtgtatcattagagtagaaaacaatccttattatcacaatttgtcaaaacaagtttgttattaacacaagtagaatactgcattcaattaagttgactgcagattttgtgttttgttaaaattagaaagagataacaacaatttgaattattgaaagtaacatgtaaatagttctacatacgttcttttgacatcttgttcaatcattgatcgaagttctttatcttggaagaatttgttccaaagactctgaaataaggaaaacaatctattatatagtctcacacctttgttttacttttagtgatttcaatttaataatgtaaatggttaaaatttattcttctctgagatcatttcacattgcagatagaaaacctgagactggggtaatttttattaaaatctaatttaatctcagaaacacatctttattctaacatcaatttttccagtttgatattatcatataaagtcagccttcctcatctgcaggttccacaacaaaaatccaaccaactgtggatcaaaaatattgggaaaaaattaaaaatagcaatacaacaataaaaaaatacaaatcagaaaaacagcacagtataacaactttatttagcatttacaatctattaggtattataagtaatctagccagatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttggacacaagacaggcttgcgagatatgtttgagaataccactttatcccgcgtcagggagaggcagtgcgtaaaaagacgcggactcatgtgaaatactggtttttagtgcgccagatctctataatctcgcgcaacctattttcccctcgaacactttttaagccgtagataaacaggctgggacacttcacatgagcgaaaaatacatcgtcacctgggacatgttgcagatccatgcacgtaaactcgcaagccgactgatgccttctgaacaatggaaaggcattattgccgtaagccgtggcggtctgtaccgggtgcgttactggcgcgtgaactgggtattcgtcatgtcgataccgtttgtatttccagctacgatcacgacaaccagcgcgagcttaaagtgctgaaacgcgcagaaggcgatggcgaaggcttcatcgttattgatgacctggtggataccggtggtactgcggttgcgattcgtgaaatgtatccaaaagcgcactttgtcaccatcttcgcaaaaccggctggtcgtccgctggttgatgactatgttgttgatatcccgcaagatacctggattgaacagccgtgggatatgggcgtcgtattcgtcccgccaatctccggtcgctaatcttttcaacgcctggcactgccgggcgttgttctttttaacttcaggcgggttacaatagtttccagtaagtattctggaggctgcatccatgacacaggcaaacctgagcgaaaccctgttcaaaccccgctttaaacatcctgaaacctcgacgctagtccgccgctttaatcacggcgcacaaccgcctgtgcagtcggcccttgatggtaaaaccatccctcactggtatcgcatgattaaccgtctgatgtggatctggcgcggcattgacccacgcgaaatcctcgacgtccaggcacgtattgtgatgagcgatgccgaacgtaccgacgatgatttatacgatacggtgattggctaccgtggcggcaactggatttatgagtgggccccggatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgcctttaatgaggaaaacctgttttgctcagaagaaatgccatctagtgatgatgaggctactgctgactctcaacattctactcctccaaaaaagaagagaaaggtagaagaccccaaggactttccttcagaattgctaagttttttgagtcatgctgtgtttagtaatagaactcttgcttgctttgctatttacaccacaaaggaaaaagctgcactgctatacaagaaaattatggaaaaatattctgtaacctttataagtaggcataacagttataatcataacatactgttttttcttactccacacaggcatagagtgtctgctattaataactatgctcaaaaattgtgtacctttagctttttaatttgtaaaggggttaataaggaatatttgatgtatagtgccttgactagagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcaactggataactcaagctaaccaaaatcatcccaaacttcccaccccataccctattaccactgccaattacctagtggtttcatttactctaaacctgtgattcctctgaattattttcattttaaagaaattgtatttgttaaatatgtactacaaacttagtagttggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgccgaattcacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccactttggctgatacgcggatctacgcgtggatccgcggcccatcacaagtttgtacaaaaaagcaggctccatttaaatctcccccatcccagcagcaagagcttgctaatcttttagctactaatcttttagccactaatctgatttccaaactgttggcacctgagctatttataaagcagtatttcatccccccagaagcctgttcttctcccctgacccccaccaatctaaaaactcagaggaccttgggtataagaggttgggcaggcgagcatagcaaccagagctggagacggatgtgagcttcatcttactccccctcgaggccaccatgaccgcctgcgcccgccgagcgggtgggcttccggaccccgggctctgcggtcccgcgtggtgggctccgtccctgccccgcctcccccgggccctgccccggctcccgctcctgctgctcctgcttctgctgcagccccccgccctctccgccgtgttcacggtgggggtcctgggcccctgggcttgcgaccccatcttctctcgggctcgcccggacctggccgcccgcctggccgccgcccgcctgaaccgcgaccccggcctggcaggcggtccccgcttcgaggtagcgctgctgcccgagccttgccggacgccgggctcgctgggggccgtgtcctccgcgctggcccgcgtgtcgggcctcgtgggtccggtgaaccctgcggcctgccggccagccgagctgctcgccgaagaagccgggatcgcgctggtgccctggggctgcccctggacgcaggcggagggcaccacggcccctgccgtgacccccgccgcggatgccctctacgccctgcttcgcgcattcggctgggcgcgcgtggccctggtcaccgccccccaggacctgtgggtggaggcgggacgctcactgtccacggcactcagggcccggggcctgcctgtcgcctccgtgacttccatggagcccttggacctgtctggagcccgggaggccctgaggaaggttcgggacgggcccagggtcacagcagtgatcatggtgatgcactcggtgctgctgggtggcgaggagcagcgctacctcctggaggccgcagaggagctgggcctgaccgatggctccctggtcttcctgcccttcgacacgatccactacgccttgtccccaggcccggaggccttggccgcactcgccaacagctcccagcttcgcagggcccacgatgccgtgctcaccctcacgcgccactgtccctctgaaggcagcgtgctggacagcctgcgcagggctcaagagcgccgcgagctgccctctgacctcaatctgcagcaggtctccccactctttggcaccatctatgacgcggtcttcttgctggcaaggggcgtggcagaagcgcgggctgccgcaggtggcagatgggtgtccggagcagctgtggcccgccacatccgggatgcgcaggtccctggcttctgcggggacctaggaggagacgaggagcccccattcgtgctgctagacacggacgcggcgggagaccggctttttgccacatacatgctggatcctgcccggggctccttcctctccgccggtacccggatgcacttcccgcgtgggggatcagcacccggacctgacccctcgtgctggttcgatccaaacaacatctgcggtggaggactggagccgggcctcgtctttcttggcttcctcctggtggttgggatggggctggctggggccttcctggcccattatgtgaggcaccggctacttcacatgcaaatggtctccggccccaacaagatcatcctgaccgtggacgacatcacctttctccacccacatgggggcacctctcgaaaggtggcccaggggagtcgatcaagtctgggtgcccgcagcatgtcagacattcgcagcggccccagccaacacttggacagccccaacattggtgtctatgagggagacagggtttggctgaagaaattcccaggggatcagcacatagctatccgcccagcaaccaagacggccttctccaagctccaggagctccggcatgagaacgtggccctctacctggggcttttcctggctcggggagcagaaggccctgcggccctctgggagggcaacctggctgtggtctcagagcactgcacgcggggctctcttcaggacctcctcgctcagagagaaataaagctggactggatgttcaagtcctccctcctgctggaccttatcaagggaataaggtatctgcaccatcgaggcgtggctcatgggcggctgaagtcacggaactgcatagtggatggcagattcgtactcaagatcactgaccacggccacgggagactgctggaagcacagaaggtgctaccggagcctcccagagcggaggaccagctgtggacagccccggagctgcttagggacccagccctggagcgccggggaacgctggccggcgacgtctttagcttggccatcatcatgcaagaagtagtgtgccgcagtgccccttatgccatgctggagctcactcccgaggaagtggtgcagagggtgcggagcccccctccactgtgtcggcccttggtgtccatggaccaggcacctgtcgagtgtatcctcctgatgaagcagtgctgggcagagcagccggaacttcggccctccatggaccacaccttcgacctgttcaagaacatcaacaagggccggaagacgaacatcattgactcgatgcttcggatgctggagcagtactctagtaacctggaggatctgatccgggacagcacggaggagctggagctggaaaagcagaagacagaccggctgcttacacagatgctgcctccgtctgtggctgaggccttgaagacggggacaccagtggagcccgagtactttgagcaagtgacactgtactttagtgacattgtgggcttcaccaccatctctgccatgagtgagcccattgaggttgtggacctgctcaacgatctctacacactctttgatgccatcattggttcccacgatgtctacaaggtggagacaataggggacgcctatatggtggcctcggggctgccccagcggaatgggcagcgacacgcggcagagatcgccaacatgtcactggacatcctcagtgccgtgggcactttccgcatgcgccatatgcctgaggttcccgtgcgcatccgcataggcctgcactcgggtccatgcgtggcaggcgtggtgggcctcaccatgccgcggtactgcctgtttggggacacggtcaacaccgcctcgcgcatggagtccaccgggctgccttaccgcatccacgtgaacttgagcactgtggggattctccgtgctctggactcgggctaccaggtggagctgcgaggccgcacggagctgaagggcaagggcgccgaggacactttctggctagtgggcagacgcggcttcaacaagcccatccccaaaccgcctgacctgcaaccggggtccagcaaccacggcatcagcctgcaggagatcccacccgagcggcgacggaagctggagaaggcgcggccgggccagttctcttgaggtgggcgcgccgacccagctttc

SEQ ID NO: 8 - DNA, 13148 bp, Artificial Sequence
  Lentiviral vector plasmid sequence Pt71 (long)
ttgtacaaagtggtgatgggtcaccatctagatggccatatgatcgatacgtactagtctcgacggtacctttaagaccaatgacttacaaggcagctgtagatcttagccactttttaaaagaaaaggggggactggaagggctaattcactcccaacgaagacaaaatcgtcgagagatgctgcatataagcagctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggccttgacattataatagatttagcaggaattgaactaggagtggagcacacaggcaaagctgcagaagtacttggaagaagccaccagagatactcacgattctgcacatacctggctaatcccagatcctaaggattacattaagtttactaacatttatataatgatttatagtttaaagtataaacttatctaatttactattctgacagatattaattaatcctcaaatatcataagagatgattactattatccccatttaacacaagaggaaactgagagggaaagatgttgaagtaattttcccacaattacagcatccgttagttacgactctatgatcttctgacacaaattccatttactcctcaccctatgactcagtcgaatatatcaaagttatggacattatgctaagtaacaaattacccttttatatagtaaatactgagtagattgagagaagaaattgtttgcaaacctgaatagcttcaagaagaagagaagtgaggataagaataacagttgtcatttaacaagttttaacaagtaacttggttagaaagggattcaaatgcataaagcaagggataaatttttctggcaacaagactatacaatataaccttaaatatgacttcaaataattgttggaacttgataaaactaattaaatattattgaagattatcaatattataaatgtaatttacttttaaaaagggaacatagaaatgtgtatcattagagtagaaaacaatccttattatcacaatttgtcaaaacaagtttgttattaacacaagtagaatactgcattcaattaagttgactgcagattttgtgttttgttaaaattagaaagagataacaacaatttgaattattgaaagtaacatgtaaatagttctacatacgttcttttgacatcttgttcaatcattgatcgaagttctttatcttggaagaatttgttccaaagactctgaaataaggaaaacaatctattatatagtctcacacctttgttttacttttagtgatttcaatttaataatgtaaatggttaaaatttattcttctctgagatcatttcacattgcagatagaaaacctgagactggggtaatttttattaaaatctaatttaatctcagaaacacatctttattctaacatcaatttttccagtttgatattatcatataaagtcagccttcctcatctgcaggttccacaacaaaaatccaaccaactgtggatcaaaaatattgggaaaaaattaaaaatagcaatacaacaataaaaaaatacaaatcagaaaaacagcacagtataacaactttatttagcatttacaatctattaggtattataagtaatctagccagatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttggacacaagacaggcttgcgagatatgtttgagaataccactttatcccgcgtcagggagaggcagtgcgtaaaaagacgcggactcatgtgaaatactggtttttagtgcgccagatctctataatctcgcgcaacctattttcccctcgaacactttttaagccgtagataaacaggctgggacacttcacatgagcgaaaaatacatcgtcacctgggacatgttgcagatccatgcacgtaaactcgcaagccgactgatgccttctgaacaatggaaaggcattattgccgtaagccgtggcggtctgtaccgggtgcgttactggcgcgtgaactgggtattcgtcatgtcgataccgtttgtatttccagctacgatcacgacaaccagcgcgagcttaaagtgctgaaacgcgcagaaggcgatggcgaaggcttcatcgttattgatgacctggtggataccggtggtactgcggttgcgattcgtgaaatgtatccaaaagcgcactttgtcaccatcttcgcaaaaccggctggtcgtccgctggttgatgactatgttgttgatatcccgcaagatacctggattgaacagccgtgggatatgggcgtcgtattcgtcccgccaatctccggtcgctaatcttttcaacgcctggcactgccgggcgttgttctttttaacttcaggcgggttacaatagtttccagtaagtattctggaggctgcatccatgacacaggcaaacctgagcgaaaccctgttcaaaccccgctttaaacatcctgaaacctcgacgctagtccgccgctttaatcacggcgcacaaccgcctgtgcagtcggcccttgatggtaaaaccatccctcactggtatcgcatgattaaccgtctgatgtggatctggcgcggcattgacccacgcgaaatcctcgacgtccaggcacgtattgtgatgagcgatgccgaacgtaccgacgatgatttatacgatacggtgattggctaccgtggcggcaactggatttatgagtgggccccggatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgcctttaatgaggaaaacctgttttgctcagaagaaatgccatctagtgatgatgaggctactgctgactctcaacattctactcctccaaaaaagaagagaaaggtagaagaccccaaggactttccttcagaattgctaagttttttgagtcatgctgtgtttagtaatagaactcttgcttgctttgctatttacaccacaaaggaaaaagctgcactgctatacaagaaaattatggaaaaatattctgtaacctttataagtaggcataacagttataatcataacatactgttttttcttactccacacaggcatagagtgtctgctattaataactatgctcaaaaattgtgtacctttagctttttaatttgtaaaggggttaataaggaatatttgatgtatagtgccttgactagagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcaactggataactcaagctaaccaaaatcatcccaaacttcccaccccataccctattaccactgccaattacctagtggtttcatttactctaaacctgtgattcctctgaattattttcattttaaagaaattgtatttgttaaatatgtactacaaacttagtagttggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgccgaattcacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccactttggctgatacgcggatctacgcgtggatccgcggcccatcacaagtttgtacaaaaaagcaggctccatttaaatcttgttaccttcctctccaacaagttcaagtctcacagatgtgcacactcagctcaatacactcagcctcccctcccccatcccacccccattgacaggagattgactcctgctgtgcacataagctgggataatgggggggggcactttctaaacatcgcttcaacagtcccaagtcccgagtagtggggggctggggaaggggtgctctttcccatacccttggcttttgtgtggcctgtaatacctgatcaagagatatagaaagactggagtgtgacactaggctctcctttcagaatcagaaagtccaatgctttgagccctcctatttctatccttcaccttgcttttcttttaatgtccctgactcgcctttgatctctggccctcaggttcaaggcctcaaaaggccaaaaccctgtagttacccttctcaagctcctctgactttaacaccatcagattcaaccactgacccctcccccatcccagcagcaagagcttgctaatcttttagctactaatcttttagccactaatctgatttccaaactgttggcacctgagctatttataaagcagtatttcatccccccagaagcctgttcttctcccctgacccccaccaatctaaaaactcagaggaccttgggtataagaggttgggcaggcgagcatagcaaccagagctggagacggatgtgagcttcatcttactccccctcgaggccaccatgaccgcctgcgcccgccgagcgggtgggcttccggaccccgggctctgcggtcccgcgtggtgggctccgtccctgccccgcctcccccgggccctgccccggctcccgctcctgctgctcctgcttctgctgcagccccccgccctctccgccgtgttcacggtgggggtcctgggcccctgggcttgcgaccccatcttctctcgggctcgcccggacctggccgcccgcctggccgccgcccgcctgaaccgcgaccccggcctggcaggcggtccccgcttcgaggtagcgctgctgcccgagccttgccggacgccgggctcgctgggggccgtgtcctccgcgctggcccgcgtgtcgggcctcgtgggtccggtgaaccctgcggcctgccggccagccgagctgctcgccgaagaagccgggatcgcgctggtgccctggggctgcccctggacgcaggcggagggcaccacggcccctgccgtgacccccgccgcggatgccctctacgccctgcttcgcgcattcggctgggcgcgcgtggccctggtcaccgccccccaggacctgtgggtggaggcgggacgctcactgtccacggcactcagggcccggggcctgcctgtcgcctccgtgacttccatggagcccttggacctgtctggagcccgggaggccctgaggaaggttcgggacgggcccagggtcacagcagtgatcatggtgatgcactcggtgctgctgggtggcgaggagcagcgctacctcctggaggccgcagaggagctgggcctgaccgatggctccctggtcttcctgcccttcgacacgatccactacgccttgtccccaggcccggaggccttggccgcactcgccaacagctcccagcttcgcagggcccacgatgccgtgctcaccctcacgcgccactgtccctctgaaggcagcgtgctggacagcctgcgcagggctcaagagcgccgcgagctgccctctgacctcaatctgcagcaggtctccccactctttggcaccatctatgacgcggtcttcttgctggcaaggggcgtggcagaagcgcgggctgccgcaggtggcagatgggtgtccggagcagctgtggcccgccacatccgggatgcgcaggtccctggcttctgcggggacctaggaggagacgaggagcccccattcgtgctgctagacacggacgcggcgggagaccggctttttgccacatacatgctggatcctgcccggggctccttcctctccgccggtacccggatgcacttcccgcgtgggggatcagcacccggacctgacccctcgtgctggttcgatccaaacaacatctgcggtggaggactggagccgggcctcgtctttcttggcttcctcctggtggttgggatggggctggctggggccttcctggcccattatgtgaggcaccggctacttcacatgcaaatggtctccggccccaacaagatcatcctgaccgtggacgacatcacctttctccacccacatgggggcacctctcgaaaggtggcccaggggagtcgatcaagtctgggtgcccgcagcatgtcagacattcgcagcggccccagccaacacttggacagccccaacattggtgtctatgagggagacagggtttggctgaagaaattcccaggggatcagcacatagctatccgcccagcaaccaagacggccttctccaagctccaggagctccggcatgagaacgtggccctctacctggggcttttcctggctcggggagcagaaggccctgcggccctctgggagggcaacctggctgtggtctcagagcactgcacgcggggctctcttcaggacctcctcgctcagagagaaataaagctggactggatgttcaagtcctccctcctgctggaccttatcaagggaataaggtatctgcaccatcgaggcgtggctcatgggcggctgaagtcacggaactgcatagtggatggcagattcgtactcaagatcactgaccacggccacgggagactgctggaagcacagaaggtgctaccggagcctcccagagcggaggaccagctgtggacagccccggagctgcttagggacccagccctggagcgccggggaacgctggccggcgacgtctttagcttggccatcatcatgcaagaagtagtgtgccgcagtgccccttatgccatgctggagctcactcccgaggaagtggtgcagagggtgcggagcccccctccactgtgtcggcccttggtgtccatggaccaggcacctgtcgagtgtatcctcctgatgaagcagtgctgggcagagcagccggaacttcggccctccatggaccacaccttcgacctgttcaagaacatcaacaagggccggaagacgaacatcattgactcgatgcttcggatgctggagcagtactctagtaacctggaggatctgatccgggacagcacggaggagctggagctggaaaagcagaagacagaccggctgcttacacagatgctgcctccgtctgtggctgaggccttgaagacggggacaccagtggagcccgagtactttgagcaagtgacactgtactttagtgacattgtgggcttcaccaccatctctgccatgagtgagcccattgaggttgtggacctgctcaacgatctctacacactctttgatgccatcattggttcccacgatgtctacaaggtggagacaataggggacgcctatatggtggcctcggggctgccccagcggaatgggcagcgacacgcggcagagatcgccaacatgtcactggacatcctcagtgccgtgggcactttccgcatgcgccatatgcctgaggttcccgtgcgcatccgcataggcctgcactcgggtccatgcgtggcaggcgtggtgggcctcaccatgccgcggtactgcctgtttggggacacggtcaacaccgcctcgcgcatggagtccaccgggctgccttaccgcatccacgtgaacttgagcactgtggggattctccgtgctctggactcgggctaccaggtggagctgcgaggccgcacggagctgaagggcaagggcgccgaggacactttctggctagtgggcagacgcggcttcaacaagcccatccccaaaccgcctgacctgcaaccggggtccagcaaccacggcatcagcctgcaggagatcccacccgagcggcgacggaagctggagaaggcgcggccgggccagttctcttgaggtgggcgcgccgacccagctttc

SEQ ID NO: 9 - DNA, 22 bp, Artificial Sequence
  PCR forward primer for genotyping
caatttgctgagggctattgag

SEQ ID NO: 10 - DNA, 20 bp, Artificial Sequence
  PCR reverse primer for genotyping
ctgtccctgtaataaacccg

SEQ ID NO: 11 - DNA, 20 bp, Artificial Sequence
  PCR forward primer for southern blotting
agatcatcctgaccgtggac

SEQ ID NO: 12 - DNA, 20 bp, Artificial Sequence
  PCR reverse primer for southern blotting
aacaggtcgaaggtgtggtc

SEQ ID NO: 13 - protein, 1110 aa, Bovine
MTACTFLAGGLRDPGLCAPTRWSPSPPGLPPIPPRPRLRLRPPLLLLLLLPRSVLSAVFTVGVLGPWACDPIFARARPDLAARLAASRLNHAAALEGGPRFEVALLPEPCRTPGSLGAVSSALTRVSGLVGPVNPAACRPAELLAQEAGVALVPWGCPGTRAAGTTAPVVTPAADALYALLRAFRWAHVALVTAPQDLWVEAGHALSTALRARGLPVALVTSMEPSDLSGAREALRRVQDGPRVRAVIMVMHSVLLGGEEQRCLLEAAEELGLADGSLVFLPFDTLHYALSPGPDALAVLANSSQLRKAHDAVLTLTRHCPLGGSVRDSLRRAQEHRELPLDLNLQQVSPLFGTIYDSVFLLAGGVARARVAAGGGWVSGAAVARHIRDARVPGFCGALGGAEEPSFVLLDTDATGDQLFATYVLDPTQGFFHSAGTPVHFPKGGRGPGPDPSCWFDPDTICNGGVEPSVVFIGFLLVVGMGLAGAFLAHYCRHRLLHIQMVSGPNKIILTLDDITFLHPHGGNSRKVAQGSRTSLAARSISDVRSIHSQLPDYTNIGLYEGDWVWLKKFPGDRHIAIRPATKMAFSKIRELRHENVALYLGLFLAGGAGGPAAPGEGVLAVVSEHCARGSLQDLLAQRDIKLDWMFKSSLLLDLIKGIRYLHHRGVAHGRLKSRNCVVDGRFVLKVTDHGHGRLLEAQRVLPEPPSAEDQLWTAPELLRDPVLERRGTLAGDVFSLGIIMQEVVCRSAPYAMLELTPEEVVKRVQSPPPLCRPSVSIDQAPMECIQLMKQCWAEQPELRPSMDRTFELFKSINKGRKMNIIDSMLRMLEQYSSNLEDLIRERTEELELEKQKTDRLLTQMLPPSVAEALKMGTPVEPEYFEEVTLYFSDIVGFTTISAMSEPIEVVDLLNDLYTLFDAIIGSHDVYKVETIGDAYMVASGLPQRNGHRHAAEIANMALDILSAVGTFRMRHMPEVPVRIRIGLHSGPCVAGVVGLTMPRYCLFGDTVNTASRMESTGLPYRIHVNRSTVQILSALNEGFLTEVRGRTELKGKGAEETYWLVGRRGFNKPIPKPPDLQPGASNHGISLHEIPPDRRQKLEKARPGQFSGK

SEQ ID NO: 14 - DNA, 910 bp, Artificial Sequence
  GUCY2D artificial sequence used as a probe in Southern blotting
agatcatcctgaccgtggacgacatcacctttctccacccacatgggggcacctctcgaaaggtggcccaggggagtcgatcaagtctgggtgcccgcagcatgtcagacattcgcagcggccccagccaacacttggacagccccaacattggtgtctatgagggagacagggtttggctgaagaaattcccaggggatcagcacatagctatccgcccagcaaccaagacggccttctccaagctccaggagctccggcatgagaacgtggccctctacctggggcttttcctggctcggggagcagaaggccctgcggccctctgggagggcaacctggctgtggtctcagagcactgcacgcggggctctcttcaggacctcctcgctcagagagaaataaagctggactggatgttcaagtcctccctcctgctggaccttatcaagggaataaggtatctgcaccatcgaggcgtggctcatgggcggctgaagtcacggaactgcatagtggatggcagattcgtactcaagatcactgaccacggccacgggagactgctggaagcacagaaggtgctaccggagcctcccagagcggaggaccagctgtggacagccccggagctgcttagggacccagccctggagcgccggggaacgctggccggcgacgtctttagcttggccatcatcatgcaagaagtagtgtgccgcagtgccccttatgccatgctggagctcactcccgaggaagtggtgcagagggtgcggagcccccctccactgtgtcggcccttggtgtccatggaccaggcacctgtcgagtgtatcctcctgatgaagcagtgctgggcagagcagccggaacttcggccctccatggaccacaccttcgacctgtt

SEQ ID NO: 15 - DNA, 768 bp, Artificial Sequence
  pig_ARR3 promoter region
ttgttaccttcctctccaacaagttcaagtctcacagatgtgcacactcagctcaatacactcagcctcccctcccccatcccacccccattgacaggagattgactcctgctgtgcacataagctgggataatgggggggggcactttctaaacatcgcttcaacagtcccaagtcccgagtagtggggggctggggaaggggtgctctttcccatacccttggcttttgtgtggcctgtaatacctgatcaagagatatagaaagactggagtgtgacactaggctctcctttcagaatcagaaagtccaatgctttgagccctcctatttctatccttcaccttgcttttcttttaatgtccctgactcgcctttgatctctggccctcaggttcaaggcctcaaaaggccaaaaccctgtagttacccttctcaagctcctctgactttaacaccatcagattcaaccactgacccctcccccatcccagcagcaagagcttgctaatcttttagctactaatcttttagccactaatctgatttccaaactgttggcacctgagctatttataaagcagtatttcatccccccagaagcctgttcttctcccctgacccccaccaatctaaaaactcagaggaccttgggtataagaggttgggcaggcgagcatagcaaccagagctggagacggatgtgagcttcatcttactccccaggtaacggtccaaagtcctctctttccagcccatactaggtcc

SEQ ID NO: 16 - DNA, 771 bp, Artificial Sequence
  Human_ARR3 promoter region
tatattaccttcctctccaatgagaagagaagttcaaggctcacagacatgtgcatacacagctcaatgcactcagatccccctccaccactcctgcccccactacctacaggagattgactcctgctgtgcacataagctgggataatcagggtttctaaacatcagcttcaaaagtccaatgtcccaaagtggtggggggctggggacgaggtactctttcccatacccttggcttttgtgtggcctggagccgctgatatagagattggagtgggacacgaggtattcctttcaaaaacacaaaggcctatactttgagccctcccatttcaatcccccaccatgcttcacctttaagacctccaactccactttgatcccagttctcaggttcaaggcctcacaaggccaaaatcctgaagttacccttctcaaactcccttgcctttaacatcatcagaatcaacctcctacccccactctgtcccagcagcaatagcctgctaatcttttagccactaatcttttaggcactaatctgctttccaaactcttggcacctgaactatttatagcagtgttttatgcccccccaccaagaaccctattcttttcccatgacccccaccaatcaaaacactcagaggactgtgggtataagaggctggggaggcaggcatagcaaccagagctggagactgatgtgaacttcatctctctccccaggtaatattccaaagccctctacttctagcccatactaagccc

SEQ ID NO: 17 - DNA, 1578 bp, Artificial Sequence
  Mouse_ARR3 promoter region
gcagagaacctgcctctaggccaggaatgctatgataccttcctttctgataagaaaaccaagtcaaggatctctactcctgccccatcacctacagaagattggttcctgctgctcactggagctgagagaatcagggtttctgattgacatcaggctgcaagagtccaaagtctcaagtgggggacaatcgggaaaggctactctttcccatgctcttggctttcttgtggcctggaaaattccagaggctagaatgtgacacaagttgctcagacaaaccaaggtccacactgtgaaccctaatagttttaccctgtgttatacttcacctctatgaatttcaacttgactttgatcccaaccctcatgtgcaatgttttcacaagtccaaaacccaccaatttccttcttttttattagatattttctttatttacatttcaaatgttatcccctttcctggtttcccctctgaaaatcccctaccccctccccactccccgtgctcaccaacccacccactccctcttcctggccctggcattcccctatactgggacatagaaccttcacaggaccaagggcctctcctcccattgatgactgactaggccatcctctgctacataggtggctggagcctgagtccctccttgtgtactctttggttggtggtttactctgggggtactggttagttcgtattgttgttcctcctaggggactgcaaaccccttcagctccttgggtcctttctctagttccttctttggggaccctgtgctcagttcaatggatggccaatttccttcttaaatgcccctagcagtaactgttaggtctcaatcccaagacaaatgtctgaggtgcctatttaacagatcaaagcggacctggcctcaggttatcccagtccctccctgtacctcagtccctacccatcaccaactctccagcccagagcttgggctgcacttcccccacggttcttcccattttggctacatggtctttttttttacctttttggttcctttggccttttggcttttggcttccagggcttctggatcccccccaacccctcccatacacatacacatgtgcactcgtgcactcaacccagcacaggataatgttcattcttgacctttccacatacatctggctatgttctctctcttatctacaataaatctcctccactatacttaggagcagttatgttcttcttctttctttcttttttttttttttcattcagtaacatcatcagaatcccctagctctggcctacctcctcagtaacaatcagctgatccctggccactaatctgtactcactaatctgttttccaaactcttggcccctgagctaattatagcagtgcttcatgccacccaccccaaccctattcttgttctctgactcccactaatctacacattcagaggattgtggatataagaggctgggaggccagcttagcaaccagagctggaggctgatgcgagcttcatctcttccctcaggtaatattctaaatctctctgcttctagcccatactaagtcc

SEQ ID NO: 18 - protein, 46 aa, Homo sapiens
IIDSMLRMLEQYSSNLEDLIRERTEELELEKQKTDRLLTQMLPPSV

SEQ ID NO: 19 - DNA, 20 bp, Artificial Sequence
  Pig GAPDH forward primer
gatggtgaaggtcggagtga

SEQ ID NO: 20 - DNA, 20 bp, Artificial Sequence
  Pig GAPDH reverse primer
aggcattgctgacgatcttg

SEQ ID NO: 21 - DNA, 19 bp, Artificial Sequence
  Pig GUCY 2D forward primer
gaggacctgatcggggagc

SEQ ID NO: 22 - DNA, 22 bp, Artificial Sequence
  Pig GUCY 2D reverse primer
caccttgtagacatcatgggag

SEQ ID NO: 23 - DNA, 19 bp, Artificial Sequence
  Human mutant GUCY 2D forward primer
gaggatctgatccgggaca

SEQ ID NO: 24 - DNA, 20 bp, Artificial Sequence
  Human mutant GUCY 2D reverse primer
tctccaccttgtagacatcg

(19) United States
(12) Patent Application Publication
(10) Pub. No.: US 2014/0033333 A1
(43) Pub. Date: Jan. 30, 2014

(54) TRANSGENIC PIG FOR MUTANT GUCY2D AS CONE DYSTROPHY MODEL

(71) Applicants: Chamsy Sarkis, Boulogne Billancourt (FR); Christopher Bruce Alexander Whitelaw, Biggar (GB); Simon Lillico, Midlothian (GB); Corinne Kostic, Chigny (CH); Yvan Arsenijevic, Carouge (CH)

(72) Inventors: Chamsy Sarkis, Boulogne Billancourt (FR); Christopher Bruce Alexander Whitelaw, Biggar (GB); Simon Lillico, Midlothian (GB); Corinne Kostic, Chigny (CH); Yvan Arsenijevic, Carouge (CH)

(73) Assignees: CENTRE NATIONAL DE LA RECHERCHE SCIENTIFIQUE, PARIS (FR); THE UNIVERSITY COURT OF THE UNIVERSITY OF EDINBURGH, EDINBURGH (GB); UNIVERSITE PIERRE ET MARIE CURIE (PARIS 6), PARIS (FR); ASILE DES AVEUGLES, LAUSANNE (CH)

(21) Appl. No.: 13/981,740

(22) PCT Filed: Jan. 27, 2012
(86) PCT No.: PCT/EP2012/051311

§ 371 Date: Oct. 18, 2013

(30) Foreign Application Priority Data
Jan. 27, 2011 (EP) 11305084.3

(51) Int. Cl. A01K 67/027

(52) U.S. Cl. CPC A01K 67/0275
USPC 800/3; 800/9; 435/325; 435/317.1; 800/21

(57) ABSTRACT

The present invention relates to a transgenic pig as a model for studying a cone affecting disease, in particular a cone dystrophy or cone-rod-dystrophy, wherein the pig model expresses a dominant negative guanylate-cyclase-2D (GUCY2D) protein, in particular a GUCY2D protein comprising at least one mutation responsible for the appearance of a CORD6 cone dystrophy in a human being. The invention further relates to methods by which the transgenic pig is produced, to uses of said transgenic pig or of one of its elements to identify new biomarkers of a cone affecting disease and/or new compounds for preventing or treating such a disease. Novel methods for preventing or treating a cone affecting disease or for evaluating conditions needed to alleviate such a disease are further herein described.

The present invention relates to a transgenic pig as a model for studying a cone affecting disease, in particular a cone dystrophy or cone-rod-dystrophy, wherein the pig model expresses a dominant negative guanylate-cyclase-2D (GUCY2D) protein, in particular a GUCY2D protein comprising at least one mutation responsible for the appearance of a CORD6 cone dystrophy in a human being. The mutation is typically present in the region located between residues 816 and 861 of SEQ ID NO:2.

The invention further relates to methods by which the transgenic pig is produced, to uses of said transgenic pig or of one of its elements to identify new biomarkers of a cone affecting disease and/or new compounds for preventing or treating such a disease. Novel methods for preventing or treating a cone affecting disease or for evaluating conditions needed to alleviate such a disease are further herein described.

### BACKGROUND OF THE INVENTION

Cone and cone-rod dystrophies (CORD) are genetically heterogeneous with described dominant, recessive, and X-linked inheritance patterns. To date, at least ten loci for autosomal dominant cone and cone-rod dystrophies have been identified: AIPL1, CRX, GUCA1A, GUCY2D, PITPNM3, PROM1, PRPH2, RIMS, SEMA4A, and UNC119.

Mutations in the GUCY2D gene coding for photoreceptor specific guanylate cyclase type1 (called ROS-GC1, retGC1 or GC-E in the literature, and herein identified as “GUCY2D protein or polypeptide”), were found in recessive forms, for patients suffering from LCA1 (Leber Congenital Amaurosis form 1), and in dominant negative forms, for patients suffering from a particular form of cone-rod dystrophy called CORD6.

Typical phenotypic characteristic of CORD6 is first deterioration of cone photoreceptors and then of rod photoreceptors. At the early stage of the disease, there is a decrease of visual acuity, a loss of color recognition and a photophobia. Electrophysiological examination shows significant loss of photopic function (cone function) without changes in scotopic function (rod function). These symptoms are the result of the dysfunction of cone photoreceptors and appear usually within the first decade of life. During the second and third decades of life, rod photoreceptor cells start to degenerate also. In addition to a severely deteriorated color vision (achromatopsia), patients develop dramatic decrease in visual acuity, night blindness and loss of peripheral vision. At the beginning of the fourth decade of life all the earlier observed symptoms are greatly increased and the electroretinogram (ERG) becomes unrecordable.

As mentioned above, GUCY2D mutations in a human being induce two distinct types of retinal disease depending on the inheritance pattern: Leber's Congenital. Amaurosis 1 for autosomal recessive mutation and CORD6 for autosomal dominant inheritance. A model of genotype/protein structure relation, appearing on FIG. 8, explains the respective consequences of these distinct mutations.

Animal models are crucial for the genetic dissection of human hereditary disease and the development of efficient treatments.

Natural animal models are already known for Retinitis Pigmentosa (RP) and for other retinal affections in mouse, rat, cat, chicken and dog.

Dogs have an area centralis that contains, in opposition to the human's one, a low proportion of cones, making it quite different from the human fovea.

Birds retina is mainly composed of cones. The development of birds retina is very different from human retina.

Mouse transgenic models of retinal diseases (Knockout (KO) mouse, Knockin (KI) mouse, mouse overexpressing a dominant negative allele), and to a lower extent rat transgenic models of retinal diseases, are known in the art. Although murine genetic models have allowed progress in the understanding of degenerative mechanisms, they do retain some major limits for the clinical evaluation of therapeutic strategies (Elizabeth Rakoczy et al., 2006).

Murines have eye and retinal anatomies which significantly differ from the human's. In particular the size of the eye is much smaller. At the retinal level, the mouse neuroretina is composed mainly of rod photoreceptors, with scattered cones barely representing 3 to 5% of the total photoreceptor population. The mouse retina can thus almost be assimilated to the human peripheral retina. In general, macular or cone degeneration is characterised by the loss of high resolution vision and by a distortion or lack of the vision at the centre of the visual field, progressing to a peripheral degeneration. In human beings in particular, macular or cone degeneration leads to difficulties in reading, impairment of face recognition, and further leads to a decrease in autonomy. To the opposite, Retinitis Pigmentosa (RP) and glaucoma are characterised by a preferential impairment of the peripheral retina in which the visual field shrinks progressively and reach “tunnel” vision. The mouse allows obtaining models of diseases affecting preferentially the peripheral retina (in other words affecting rods preferentially but not cones), and often displays phenotypes more pronounced than those observed in humans. As the murine retina does not have macula or a cone-rich area centralis, it is recognized as an inappropriate model of macular degenerations and cone-affecting diseases (Marmorstein and Marmorstein, 2007).

Therapeutic approaches tested or screened in rodents eye may be very unpredictive of what could be observed in a human being. This can be explained by discrepancies due to the difference of surgical procedures, eye size, eye pressure, immune or inflammatory responses, diffusion within the eye or drug clearance of administered compounds. It can also result from differences in the anatomy of the retina of rodents and human, in particular from differences in the distribution of cones within the retina (the human eye having a fovea containing almost exclusively cones, whereas the cones is mice are scattered within rod cells).

Adequate animal models of dominant inheritance CORD6 have not been produced until now. Only null mutants of the GUCY2D gene in mice (KO mice) or chicken (null mutant chick) constituting models of Leber Congenital Amaurosis 1 (LCA1) have been described.

The guanylate cyclase 1 (GC1) KO mouse (Coleman J E et al., 2004) is a mammalian model of Leber congenital amaurosis 1 (LCA1).

The chicken model, also a model of LCA1, carries a deletion rearrangement in the gene encoding retGC1 that produces a null allele (Semple-Rowland S L et al., 1998).

Inventors now herein provide a transgenic pig as a model for studying a cone affecting disease, in particular cone and cone-rod dystrophies, comprising a recombinant nucleic acid, encoding a dominant negative human guanylate-cyclase-2D (GUCY2D) protein or polypeptide, which is preferably stably integrated in the pig genome and which is preferably expressed in cones.

Inventors herein demonstrate that pig, which has an area centralis which is similar in many ways to the human fovea, is particularly suited to generate an animal model of cone affecting diseases mimicking human pathologies.

### SUMMARY OF THE INVENTION

Inventors developed a method for producing transgenic mice and pigs overexpressing in their cone cells a human dominant negative allele of the GUCY2D gene. Inventors demonstrate that the mouse model is not satisfying making it of poor interest for research to better understand and treat CORD6 affections in particular. In contrast, inventors demonstrate that transgenic pigs according to the present invention present fundamental characteristics of the human disease, i.e. impairment of cones, as assessed in particular by a clear reduction of cone activity, a diminution of visual acuity and signs of structural changes of the retina as further herein described.

This pig model allows a better understanding of the aetiology of cone- and cone-rod dystrophies. The transgenic pigs, their organs, tissues, cells as well as any cellular or subcellular extract can be used for identifying or validating biomarkers and therapeutic targets. This model can also be used to test preventive or therapeutic approaches including drug therapies, gene therapy, cell therapy, as well as artificial retina therapy.

As many cone-affecting diseases share common marks, results obtained with the GUCY2D pig model can be extrapolated to other cone-affecting diseases, including cone dystrophies, cone-rod dystrophies, macular diseases and age-related macular degenerations.

A transgenic pig is thus herein provided as a model for studying a cone affecting disease, in particular a cone dystrophy or cone-rod-dystrophy, as well as a genetically modified cell derived from such a transgenic pig, the nucleus of such a genetically modified cell, a pig or a population of cells derived from such a genetically modified cell. This model comprises a recombinant nucleic acid which is preferably stably integrated in its genome, and which encodes a dominant negative human guanylate-cyclase-2D (GUCY2D) protein.

Any fertilized egg, zygote, morula, blastocyst, embryo, or fetus derived from the herein described transgenic pig model also belongs to the present invention.

Further herein described is the use of a transgenic pig as herein described or of a fertilized egg, a zygote, a morula, a blastocyst, an embryo, or a fetus according the present invention for the in vivo evaluation of the ability of a compound, to prevent or treat a cone affecting disease, typically a cone- or cone-rod dystrophy, or for the screening of a compound for preventing or treating such a cone affecting disease.

Also herein described are the use of a genetically modified cell as herein described or of a population of such cells, for the in vitro or ex vivo evaluation of the ability of a test compound to prevent or treat a cone affecting disease, typically a cone- or cone-rod dystrophy, or for the screening of a compound for preventing or treating cone dystrophy, or for the in vitro or ex vivo identification of a therapeutic target usable to prevent or treat such a cone affecting disease.

Also herein provided is a method for evaluating the efficacy of a compound for preventing or treating a cone affecting disease, typically a cone- or cone-rod dystrophy, said method comprising the steps of i) providing a pig model according to the present invention, ii) administering to said pig model a compound the efficacy of which is to be evaluated, and iii) evaluating the effect, if any, of the compound on the phenotype induced by the dominant negative GUCY2D protein expressed in the pig model.

Further herein provided is a method for evaluating the efficacy of an artificial retina or of a biocompatible polymer capsule in the treatment of a cone affecting disease, typically a cone- or cone-rod dystrophy, said method comprising the steps of i) providing a pig model according to the present invention, ii) grafting to said pig model an artificial retina or a biocompatible polymer capsule the efficacy of which is to be evaluated, and iii) evaluating the effect, if any, of the artificial retina or of the biocompatible polymer capsule on the phenotype induced by the mutated GUCY2D protein expressed in the pig model of cone affecting disease.

A particularly efficient process for producing a transgenic pig according to the present invention usable as a model for studying a cone affecting disease, typically a cone- or cone-rod dystrophy, is also herein described. This process comprises the steps of:
a) providing a nucleic acid expression cassette comprising a promoter operably linked to a recombinant nucleic acid encoding a dominant negative human guanylate-cyclase-2D (GUCY2D) protein or polypeptide,
b) placing said cassette within an embryo of a female pig under conditions in which said cassette is stably integrated into the genome of said pig; and
c) causing said embryo to go to term so as to generate a transgenic pig which is a model for studying a cone affecting disease.

In a preferred embodiment, the nucleic acid expression cassette is contained in a lentiviral vector produced with a plasmid containing said expression cassette. Even more preferably, the nucleic acid expression cassette is contained in a lentiviral vector produced with a plasmid consisting in SEQ ID NO:7 or 8.

### DESCRIPTION OF DRAWINGS

FIG. 1: Alignment of the pig (SEQ ID NO: 15), human (SEQ ID NO: 16), and mouse (SEQ ID NO: 17) genomic sequences covering the region of the arrestin 3 (ARR3) promoter.

Putative CRX binding sites are boxed (full line), putative TATA-box are boxed (dotted line) and the predicted TSS transcription start site (TSS) of pig is indicated with an arrow.

FIG. 2: Lentiviral construct Pt71 (long promoter).

A lentiviral vector plasmid was used to produce lentiviral vectors comprising the GUCY2D mutant allele (E837D/R838S) under the control of the long Arrestin3 pig promoter sequence.

FIG. 3: Lentiviral construct Pt75 (short promoter).

A lentiviral vector plasmid was used to produce lentiviral vectors comprising the GUCY2D mutant allele (E837D/R838S) under the control of the short Arrestin3 pig promoter sequence.

FIG. 4: Representative images of PCR products obtained by amplification of a lentiviral backbone segment for genotyping of pigs.

PCR screening was performed with primers for the HIV1 backbone and produced a specific product when DNA from transgenic animals was used as template. Genomic DNA from a non-transgenic transgenic pig was included as a negative control. Plasmid DNA from the Pt71 lentiviral vector plasmid (SEQ ID N: 8) was used as a positive control.

FIG. 5: representative image of Southern blotting for detecting and quantifying the human GUCY2D transgene.

Southern blotting was performed with a DIG labelled fragment of the human GUCY2D gene against junction fragments generated by EcoRI-digestion of porcine gDNA. Individual transgene integration events are represented by individual bands on the Southern blot. Cross hybridization of the probe with its porcine orthologue revealed a common fragment in all samples regardless of transgene status.

FIG. 6: Picture of the visual function tests settings.

Three behavioral tests were used to assess the visual function of pigs. The first one, the “ball test” consists in moving vertically a ball at the left or right side of the pig's head, which head is restrained by two boards. The pig reaction is video recorded and classified from “surprise” to indifference.

The second test consists of a maze that was constructed in a corridor flanked by two boards. A large colored ball is visible from the maze start. The time necessary for the animals to reach the ball and pass both “doors” is measured.

At 24 weeks age, in condition of strong light (1000 to 2400 lux), a maze was constructed which consisted of a corridor were various colored obstacles (circulation cones, buckets and a suspended flying-disc) were placed at fix points. The time necessary for the animals to pass the maze was measured.

FIG. 7: Histology of transgenic pigs retina at 4 and 7 months age.

Two animals were sacrificed for histological analysis. The eyes were sectioned by both microtome and cryostat and stainings were performed to reveal retinal morphology and cone survival.

FIG. 8: Genotype/protein structure relation model (Duda and Koch. Molecular and Cellular Biochemistry 230: 129-138, 2002.).

Consequences of LCA1 (A) and CORD6 (B) mutations. In this example, the autosomal recessive point mutation F514S leads to a complete loss of guanylate cyclase-activating protein 1 (GCAP1) sensitivity. Since this mutation is located in the binding site for GCAP1 in the cytoplasmic part or juxtamembrane domain (JMD), GCAP1 probably has lost contact to this site. In this case, the autosomal recessive F514S substitution leads to the selective destruction of the phototransduction linked ROS-GC1 stimulation by GCAP1.

In the CORD6 (Example B), the triple autosomal dominant mutation inducing the substitution of E₈₃₇R₈₃₈T₈₃₉ by D₈₃₇C₈₃₈M₈₃₉ disturbs the dimerization of GUCY2D (also called ROS-GC1). Because the regulatory sites of GCAP1 are intact, this mutant responds to GCAP1 (although in a modified manner), but has a reduced basal activity. Thus, distortion of the dimer formation will be a reason for the lower basal guanylate cyclase activity, since a correct dimer interface is necessary for efficient catalysis.

Dominant mutations provoking CORD6 are thought to affect the negative retro-action by Ca++ and/or GCAP1 thus leading to an accumulation of cGMP.

FIG. 9: Representative image of RT PCR (detection of the transgene expression)

Expression of the transgene human mutant GUCY2D (A) is revealed by RT−PCR, performed of RNA extracted from retina collected on transgenic and non-transgenic animals at 18 months of age. Expression of endogenous GUCY2D (B) and GAPDH (C) were also performed as control. Control sample (SC) from spinal cord tissues of non transgenic animals were also used as well as “pt71”, a plasmid containing the human mutant GUCY2D cDNA (for A, B and C: L: DNA ladder; 1: #904 cDNA prepared with reverse transcriptase (RT+); 2: #904 cDNA control without reverse transcriptase (RT−); 3: #907 RT+; 4: #907 RT−; 5: #908 RT+; 6: #908 RT−; 7: #914 RT+; 8: #914 RT−; 9: #915 RT+; 10: #915 RT−; 11: #917 RT+; 12: #917 RT−; 13: #918 RT+; 14: #918 RT−; 15: #920 RT+; 16: #920 RT−; 17: #929 RT+; 18: #929 RT−; 19: SC RT+; 20: SC RT−; 21: H2O RT+; 22: H2O RT−; 23: empty; 24: pt71). #904, #907, #908, #914, #915, #917, #918, and #920 were shown to be transgenic animals by PCR on genomic DNA, #929 was shown to be non-transgenic animal by PCR on genomic DNA). The differences observed in the transgene expression level (as shown by PCR signal intensity differences) reflex differences in the number of copies integrated into each animal genome as well as the differences of transcriptional permissivity of the various integration site which differ among the animals.

FIG. 10: Histology of the transgenic retina from 4 to 18 months of age, quantification of cones.

Transgenic (TG) and control (WT) animals were sacrificed at 4 (WT: n=1; TG: n=1), 15 (WT: n=4; TG: n=1) and 18 (WT: n=3; TG: n=9) months of age, eyes were collected and processed for paraffin embedding. Several immunostainings were performed on paraffin sections (see FIG. 7 for representative images) and cones were quantified. The number of displaced nuclei in the outersegments (A) was determined for the entire section; the numbers of cones labeled by PNA (B) or anti-M/L-opsin (C) were determined at a particular position on the sections.

### DETAILED DESCRIPTION

As explained previously, there is a need in the art for improved tools and methods for studying cone affecting disease, in particular cone dystrophy. The present invention provides such improved tools and methods.

In the context of the present invention, cone affecting diseases include cone dystrophies, cone-rod dystrophies, macular diseases and age-related macular degenerations (AMD or ARMD).

The present invention more particularly provides a transgenic pig usable as a new research tool and usable, contrary to the animal models of the art, as a clinically relevant animal model. This new model has been used by inventors to obtain valuable information regarding the role of GUCY2D in the pathogenesis of the CORD6 cone dystrophy.

The transgenic pig according to the present invention comprises a recombinant nucleic acid encoding a dominant-negative guanylate-cyclase-2D (GUCY2D) protein or polypeptide, preferably stably integrated in its genome, in particular a dominant-negative human GUCY2D protein or polypeptide.

A “nucleic acid” according to the invention refers to polynucleotides, such as DNA, in particular cDNA, RNA, modified DNA, modified RNA, as well as mixtures thereof.

A “dominant-negative” GUCY2D protein or polypeptide according to the invention refers to a modified GUCY2D inducing high concentrations of Ca²⁺ and cGMP in photoreceptor cells responsible for the dysfunction of cone cells first and then of rod cells.

Preferably, the recombinant nucleic acid encodes a dominant-negative GUCY2D protein or polypeptide which comprises at least one mutation in the region of human GUCY2D located between residues 816 and 861 of SEQ ID NO: 2, said mutation being responsible for the appearance of a cone affecting disease, such as a CORD6 cone dystrophy, in human beings.

The region located between residues 816 and 861 of SEQ ID NO: 2 (wild-type human GUCY2D peptide sequence) comprises the following 46 amino acid residues:

(SEQ ID NO: 18)
IIDSMLRMLEQYSSNLEDLIRERTEELELEKQKTDRLLTQMLPPSV.

The mutation is typically a substitution or amino acid change, typically a non conservative substitution, of at least one residue, preferably of two or three residues, even more preferably of two residues, selected from residue 837, 838 and 939, of the region of human GUCY2D located between residues 816 and 861 of SEQ ID NO: 2. Preferred substitutions responsible for the appearance of a CORD6 cone dystrophy in a human being may be selected from a substitution of residue 837, of residue 838, and substitutions of residues 837 and 838, of 838 and 839 or of 837, 838 and 839 of SEQ ID NO: 2.

More preferably, the recombinant nucleic acid encodes a GUCY2D protein or polypeptide comprising or consisting in SEQ ID NO: 4 or in a functional analog or homolog thereof.

The amino acid sequence of SEQ ID NO: 4 comprises the E837D and the R838S mutations.

Preferably, the recombinant nucleic acid is a cDNA consisting in SEQ ID NO: 3.

The substitution at residue 837 is preferably a substitution of Glu (glutamic acid) by Asp (Aspartic acid) (E837D) in SEQ ID NO:2. In other words, E837D indicates substitution of the Glu residue (E) at position 837 of SEQ ID NO:2 by a Asp (D) residue.

The substitution at residue 839 is preferably a substitution of Thr (Threonine) by Met (Methionine) (T839M) in SEQ ID NO:2.

The substitution at residue 838, in SEQ ID NO:2, may be selected from R838S, R838C, R838D, R838H, R838E, R838K, R838L or R838A. Preferred substitutions are substitution of Arg (Arginine) by Ser (Serine) (R838S) and substitution of Arg (Arginine) by Cys (Cysteine) (R838C).

In the context of the present invention, a homolog of the human GUCY2D amino acid sequence of SEQ ID NO: 2, preferably a functional homolog, in particular a functional homolog responsible for the appearance of a cone affecting disease in a human being (in particular CORD6), may be any homologous sequence from a distinct mammal exhibiting an amino acid sequence homology of at least 70%; preferably at least 80%, even more preferably of at least 90, 95, 98 or 99% with SEQ ID NO: 2, such as the porcine GUCY2D amino acid sequence or the bovine GUCY2D amino acid sequence of SEQ ID NO: 13.

The herein used DNA sequence advantageously comprises a promoter active in the transgenic pig, in particular a promoter active in vivo and ex vivo in retinal cone cells of the transgenic pig. The promoter may be a cellular, a synthetic or a chimeric promoter.

The term “promoter” herein refers to a nucleic acid sequence comprising a minimal promoter allowing transcriptional activity in a cell together with transcriptional regulatory elements allowing adequate expression levels and cell specificity. Examples of regulatory elements include an enhancer sequence, a silencer sequence, a 5′UTR sequence, and an intron such as the first intron of the downstream recombinant nucleic acid sequence to be expressed.

This promoter is preferably selected from a porcine promoter and any homolog thereof, preferably functional homolog (as previously defined) capable of allowing the expression of recombinant nucleic acid of interest in retinal cone cells of a transgenic pig, in particular a human, mouse, rat or bovine promoter. More preferably, the promoter contains the binding sites specific for the transcription factors controlling specific cone gene expression.

The promoter is advantageously selected from an arrestin promoter; a blue opsin promoter (Komaromy et al., 2008, Gene. Ther. 15, 1049-1055), a red opsin promoter (Komaromy et al.), and a green opsin promoter.

Inventors herein demonstrate that the arrestine promoter advantageously allows the expression of a transgene in cone cells. Inventors herein confirm that the arrestine promoter advantageously allows the expression, in retinal cone cells, of a GUCY2D protein or polypeptide encoded by any one of the herein described nucleic acid sequences.

The promoter may be selected from the short cone Arrestin promoter of SEQ ID NO: 5, the long cone Arrestine promoter of SEQ ID NO: 6 and any homolog, preferably functional homolog, thereof. Preferably, the promoter is the long cone Arrestine promoter of SEQ ID NO: 6.

Herein described are methods for preparing a transgenic pig with a cone affecting disease, in particular with cone dystrophy.

A particular process for producing a transgenic pig according to the present invention, usable as a model for studying a cone affecting disease, comprises the steps of:
a) providing a nucleic acid expression cassette comprising a promoter as herein described, in particular an arrestine promoter, operably linked to a recombinant nucleic acid encoding a dominant negative human guanylate-cyclase-2D (GUCY2D) protein (or polypeptide),
b) placing said cassette within an embryo of a female pig under conditions in which said cassette is stably integrated into the genome of said pig; and
c) causing said embryo to go to term so as to generate a transgenic pig which is a model for studying a cone affecting disease.

Preferably, the nucleic acid expression cassette is a plasmid or is contained in a plasmid. This plasmid may be placed directly within an embryo or may be used to prepare a viral vector, preferably a lentiviral vector, which will be placed within the embryo in the context of the method as herein described for preparing the transgenic pig of the invention.

A preferred viral vector is a retroviral vector, preferably a lentiviral vector, in particular a HIV-derived retroviral vector, typically a HIV-1-derived retroviral vector, preferably a lentiviral vector prepared or produced with a plasmid consisting in SEQ ID NO: 7 or 8.

A lentiviral vector advantageously usable in the context of the present invention comprises HIV retroviral GAG and POL proteins, an heterologous ENV protein, preferably a Vesicular stomatitis virus (VSV) ENV protein, a retroviral genome comprising the recombinant nucleic acid sequence encoding the GUCY2D amino acid sequence of interest operably linked to a regulatory sequence, preferably to a promoter sequence as herein described.

HIV retroviral cis-acting nucleic acid sequences typically comprise:
Long terminal Repeat (LTR) sequences, preferably HIV-LTR sequences, preferably comprising a self-inactivating (SIN) lentiviral 3′LTR;
a cis-acting nucleic acid sequence facilitating the RNA nuclear export, preferably the HIV-1 rev Responsive Element (RRE),
preferably one copy of the cPPT and CTS cis-acting regions (“flap sequence”) of HIV-1,
an HIV retroviral packaging nucleic acid sequence comprising an HIV retroviral 5′ splice donor sequence, and
a psi sequence.

The self-inactivating (SIN) lentiviral 3′LTR may be:
a 3′LTR deleted from the U3 region,
a 3′LTR deleted from the enhancer sequence of the U3 region, or
a 3′LTR deleted from the enhancer and promoter sequences of the U3 region.

The viral vector is preferably produced in a packaging host cell containing:
at least one, possibly two or more, transcomplementation plasmid(s) providing nucleic acid sequences linked to a heterologous regulatory nucleic acid sequence that respectively encode the HIV retroviral GAG, POL, TAT and REV proteins, and linked to an heterologous polyadenylation signal;
an envelope plasmid providing a nucleic acid encoding a heterologous ENV protein, preferably derived from VSV-G; and
an expression plasmid providing a nucleic acid sequence containing preferably an HIV retroviral packaging signal flanked by HIV retroviral cis-acting nucleic acid sequences (as described previously); a less than full length HIV gag structural gene; and the nucleic acid expression cassette comprising a promoter as herein described operably linked to a recombinant nucleic acid encoding a dominant negative human guanylate-cyclase-2D (GUCY2D) protein of interest.

The transcomplementation plasmid is preferably devoid of one or more accessory genes (vif, vpr, vpu and nef genes).

As explained previously, in a preferred aspect of the method herein described for obtaining transgenic pig according to the present invention, the nucleic acid expression cassette comprising a promoter operably linked to a recombinant nucleic acid encoding a dominant negative human guanylate-cyclase-2D (GUCY2D) protein is introduced into early embryos via a lentiviral vector.

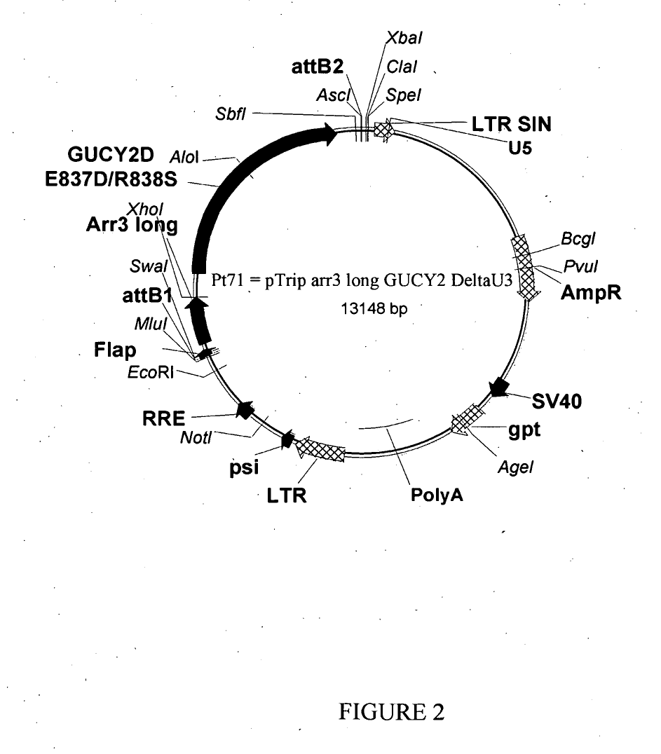

A preferred viral construct is shown on FIG. 2 and corresponds to SEQ ID NO: 8.

Any other method for introducing the expression vector into early embryos is however possible. The transgenic pig according to the present invention can be obtained by introducing the recombinant nucleic acid, preferably the previously mentioned expression cassette containing a promoter regulating its expression, into a fertilized egg or the like (clonal egg or embryo for example), by the conventional method of pronuclear injection or by the conventional sperm, vector method; and developing an individual from the fertilized egg or the like by returning the embryo to the uterus of a foster mother at an appropriate stage.

The nucleic acid to be introduced into the fertilized egg or the like is preferably linear in order to increase the probability that the nucleic acid is incorporated into the chromosomal DNA.

The “clonal egg” herein means an egg obtained by transplanting a nucleus of a somatic cell (in case of a somatic cell clone) or a fertilized egg (in case of fertilized egg clone) into an enucleated recipient egg.

The “embryo” herein means an embryo in an optimal stage between a unicellular egg and an embryo which can develop, preferentially to term, if returned to a uterus (preferably an embryo in the completely hatched blastocyst stage). However, introducing the gene in the stage of unicellular egg is preferred because the gene is incorporated in all of the cells of the transgenic pig. An individual can be developed, preferably, by growing the egg or embryo into which the gene was introduced up to the morula stage, and returning the resulting embryo to a uterus of an animal.

Alternatively, the expression vector can be introduced into somatic cells which will then be used for nuclear transfer to generate a cloned transgenic animal according to the conventional somatic cell nuclear transfer method (Gil M A et al., 2010, Reprod Domest Anim. 2010 June; 45 Suppl 2:40-8).

Other objects of the present invention are a transgenic pig, including a transgenic pig obtainable by a method as herein described, at the various stage of its formation and development (fertilized egg, zygote, morula, blastocyst, embryo, fetus, young pig or adult pig), as well as its elements, typically an isolated element, and its progeny. The pig may be alive or not. The pig or any of its elements can be frozen using any method known by the skilled person.

The transgenic pig is advantageously a domestic pig. The domestic pig can be selected for example from a miniature pig, a minipig and a micropig.

The transgenic pig according to the present invention preferably contains the recombinant nucleic acid encoding GUCY2D in its somatic cells, in particular in ocular cells, typically in retinal cells, and/or in its germ cells.

Elements of the transgenic pig comprise in particular a genetically modified cell or tissue, i.e., a genetically modified cell or tissue derived from said transgenic pig, for example a cell expressing GUCY2D, typically a retinal cell; any cellular or sub-cellular extract of such a genetically modified cell such as the nucleus, a protein, a nucleic acid in particular a DNA or RNA, an organelle; a population of genetically modified cells directly obtained (sampled) from the transgenic pig or derived (cultured) from an isolated genetically modified cell as described previously, typically a cell line.

The genetically modified cell can be selected from a stem cell, in particular an induced pluripotent stem cell (iPS cell), a germ cell, a gamete and a somatic cell.

The skilled person is able to determine suitable methods and procedure for obtaining transgenic cells from the transgenic pigs or cell lines from said transgenic cells.

The transgenic cells and/or cell lines are suitable in vitro test systems and can be used for developing autologous or xenologous cell replacement therapies.

Transgenic cell lines can in particular be established in order to generate a standardized model system for cone affecting disease research.

The transgenic cells and/or cell lines, preferably the transgenic somatic cells and/or cell lines, can also be used to obtain transgenic pigs, such as by cloning strategies. Further herein enclosed is therefore a pig derived from a genetically modified cell as herein described.

The term “progeny” indeed herein includes not only the progeny obtained by the normal sexual reproduction, but also the pigs cloned from somatic cells having the same chromosomal DNA as the transgenic pigs herein described (produced by the conventional somatic cell nuclear transfer cloning technique or any other suitable method that the skilled person can use to generate cloned pigs) which contain the recombinant nucleic acid encoding a dominant negative GUCY2D integrated in their chromosomal DNA.

Preferably, the transgenic pig or its progeny expresses at least one of the following detectable and/or measurable features or phenotypes associated with cone dystrophy:
a decrease of cones electrophysiological activity (as assessed for example by photopic scoring by ERG);
a modification of pig ambulatory behaviour, or in other words a decrease of pig functional visual function (as assessed for example by behavioural tests such as “obstacle course or obstacle maze” or “the ball test”);
structural changes of retina (as assessed for example by histology or optical coherence tomography (OCT)) consisting in i) migration of at least part of cells, in particular of at least part of cones, ii) morphological modifications of at least part of cells, iii) in cell degeneration of at least part of cells and/or iv) in cell death of at least part of cells.

The herein described transgenic pig can advantageously be used for research studies as well as for prevention and/or treatment of a cone affecting disease, in particular of a cone dystrophy, of a cone-rod dystrophy, of a macular disease or of an age-related macular degeneration.

Preferably the transgenic pigs of the invention are used as model systems for studying the pathogenesis, i.e., the onset, development and progress, of a cone affecting disease in particular of a cone dystrophy or of a cone-rod dystrophy.

Herein described transgenic pigs can be used to evaluate the role of a dominant negative GUCY2D protein or polypeptide in the pathogenesis of cone affecting diseases, for example by profiling or characterizing signalling mechanisms depending on the correct expression of GUCY2D.

Preferably, the transgenic pigs of the invention are used as model systems for the prevention and/or treatment i.e., for identifying means and methods suitable for the prevention and/or treatment (complete cure of the disease or alleviation of the disease' symptoms), of a cone affecting disease, in particular of a cone dystrophy or of a cone-rod dystrophy.

The use of a pig as herein described as a model system for the prevention and/or treatment of a cone affecting disease typically comprises the development and evaluation of a therapeutic strategy of a cone affecting disease. Different treatment regimens can be evaluated with regard to efficacy and safety, in a continuous manner (over the lifetime or over certain period of time during the life of a transgenic pig) or in time intervals. The time intervals are preferably six-monthly, three-monthly, monthly, two weeks intervals or even weekly intervals. The skilled artisan is able to choose further suitable time intervals.

In a particular embodiment, a method for studying the pathogenesis, the prevention and/or the treatment of a cone affecting disease using a pig model according to the present invention, whatever the stage of its formation and development, or an element thereof as herein defined, is provided.

The transgenic pigs of the invention are, as explained previously, highly suitable models or model systems for a cone affecting disease, because they exhibit the previously described key features of cone as well as cone-rod-dystrophy. The transgenic pig model furthermore overcomes the previously described limitations of existing models, in particular of mouse models. For the first time, a transgenic large animal model with impaired GUCY2D function is established. This model exhibits genetic, physiologic and biochemical functions, in particular immunologic, endocrine as well as metabolic functions, more similar to those of a human being than the corresponding functions of other animal models. Also, in the respect of eating habits, pigs are omnivorous with a gastrointestinal tract resembling the human being's.

There are anatomical similarities between the human and the porcine eye, macroscopic as well as histological, making, as herein demonstrated, this animal a good model for testing ophthalmologic treatment modalities and surgical procedures. The porcine eye is in particular similar to the human eye in terms of size. The dimensions of a human eye are of about 24.0×23.5×24 mm (width×height×depth) while an eye of a pig weighing approximately 30 kg, has dimensions of about 23.0×21.0×19 mm (width×height×depth) (Kiilgaard 2002). Thus, the similarity in size makes it possible to use equipment and surgical devices developed for human purposes in the porcine eye. It also facilitates the possible transfer of new surgical techniques developed and practiced in the porcine eye to the human eye.

Histologically, the porcine retina further consists of the same ten retinal layers as the human retina. Bruch's membrane and the choroid are also similar. Both pigs and humans have a retinal as well as a choroidal circulation. Studies by Hendrickson and Hicks, 2002 and Voss et al. 2007, show that the pig retina has a high density of cones. A large horizontal band running across the retina at and above the optic disc (OD) contains the highest cone density, and corresponds to a similar streak of high ganglion cell density. This band is also free of large blood vessels. These characteristics are similar to those of the human fovea.

The distribution of cones within the retina may have strong impact on biology of the retina itself and on the effect of treatments. Indeed, the neuroretina comprises both rods and cones that can be distributed differently depending on the species, as described herein. Rods are known to secrete trophic factors or survival factors. The position and distribution of cones within the rods population may thus impact on the protective effect on cones of such secreted factors. The rod-secreted factors may affect differently cone cells if they are scattered or clustered in particular regions of the retina. In the same way, degenerating cells are known to influence the biology of nearby cells. Rods that are degenerating may thus have different effects of nearby cones, depending on their scattered (i.e. in mouse or rat) or clustered organization (i.e. in pig or human, as herein demonstrated).

It is an object of the present invention to use a transgenic pig as herein described, whatever the stage of its formation and development, for the in vivo identification of or evaluation of the ability of, a compound, also herein identified as test compound, to prevent or treat a cone affecting disease, in particular a cone dystrophy or a cone-rod dystrophy, or to use a genetically modified cell, a population of cells or a tissue comprising such genetically modified cell as herein described, for the in vitro or ex vivo evaluation of the ability of a test compound to prevent or treat a cone affecting disease, in particular a cone dystrophy or a cone-rod dystrophy.

A method for evaluating the efficacy of a compound for preventing or treating a cone affecting disease is more particularly herein described. This method comprises the steps of i) providing a pig model according to the present invention, whatever the stage of its formation and development, or an element thereof as herein defined, ii) administering to said pig model or element a compound the efficacy of which is to be evaluated, and iii) evaluating the effect, if any, of the compound on the phenotype induced by the mutated (dominant negative) GUCY2D protein or polypeptide expressed in the pig model.

It is also an object of the present invention to use of a transgenic pig as herein described, whatever the stage of its formation and development, for the screening of a compound for preventing or treating a cone affecting disease, in particular a cone dystrophy or a cone-rod dystrophy, or to use a genetically modified cell, a population of cells or a tissue comprising such genetically modified cell as herein described, for the screening of a test compound for preventing or treating such a cone affecting disease.

A screening method of the invention preferably comprises the following steps of i) providing a pig model according to the present invention, whatever the stage of its formation and development, or an element thereof as herein defined, (ii) providing a compound to be tested, (iii) administering the compound to said pig model or element, (iv) determining whether the tested compound is capable of preventing or treating a cone affecting disease as herein described.

The compound tested in vivo in a transgenic pig as herein described can be selected from a drug, a nucleic acid, a cell, a population of cells, a functional food, a therapeutic vector and any mixture thereof.

The term “drug” herein refers to a substance, a medication or pharmaceutical composition that, when administered to a living organism, is used in the treatment (preferably complete cure), prevention, or diagnosis of disease or used to otherwise enhance physical well-being.

The term “functional food” or medicinal food refers to any healthy food which has a health-promoting or disease-preventing property beyond the basic function of supplying nutrients. The term “functional food” encompasses food, food complements and mixture thereof.

Examples of food complement are a vitamin, for example vitamin A, vitamin C or vitamin E; zinc; a complement with high linolenic acid (omega-3 fatty acid) and low linoleic acid (omega-6 fatty acid); DHA (an omega-3 fatty acid); resveratrol; carotenoids, for example, lutein and zeaxanthin.

The “therapeutic vector” is a vector that allows inserting, altering or removing a gene within an individual's cell to treat a disease. Therapeutic vectors comprise any synthetic vectors, for example nucleic acids (DNA or RNA, or a mixture thereof), whether naked or complexed, for example pegylated nucleic acids or nucleic acids encapsulated into nanovectors or nanoparticles; virus derived vectors, for example adenovirus-derived vectors, retrovirus derived vectors, in particular lentivirus-derived vectors, adeno-associated-virus derived vectors, alphavirus-derived vectors and baculovirus-derived vectors; as well as mixtures thereof.

The compound tested in vitro or ex vivo in a genetically modified cell, in a population of cells or in a tissue comprising such genetically modified cell is preferably selected from a drug, a therapeutic vector, as well as mixture thereof.

It is a further object of the present invention to use a genetically modified cell, a population of cells or a tissue comprising such genetically modified cell as herein described, for the in vitro or ex vivo identification of a biomarker or therapeutic target usable to prevent or treat a cone affecting disease, such as in particular cone- and cone-rod dystrophies as well as macular diseases and age-related macular degenerations.

Another method for evaluating the efficacy, typically in the treatment of a cone affecting disease as herein described, of an artificial retina or of a biocompatible polymer capsule is further herein provided. This method comprises the steps of i) providing a pig model according to the present invention whatever the stage of its formation and development, ii) grafting to said pig model an artificial retina or a biocompatible polymer capsule the efficacy of which is to be evaluated, and iii) evaluating the effect, if any, of the artificial retina or of the biocompatible polymer capsule on the phenotype induced by the mutated GUCY2D protein expressed in the pig model.

The term “artificial retina” herein designates implantable microelectronic retinal, or epiretinal, prosthesis that restores useful vision to people affected by a retinal disease.

The term “biocompatible polymer capsule” herein designates solid, porous or hollow capsules composed of a biocompatible polymer, eventually biodegradable. Alternatively a biocompatible capsule may comprise a treating agent or cells encapsulated within a polymer shell or a polymer sphere.

Further aspects and advantages of the present invention will be described in the following examples, which should be regarded as illustrative and not limiting.

### EXPERIMENTAL PART

### Example 1

### Porcine Cone Arrestin-3 Promoter Design

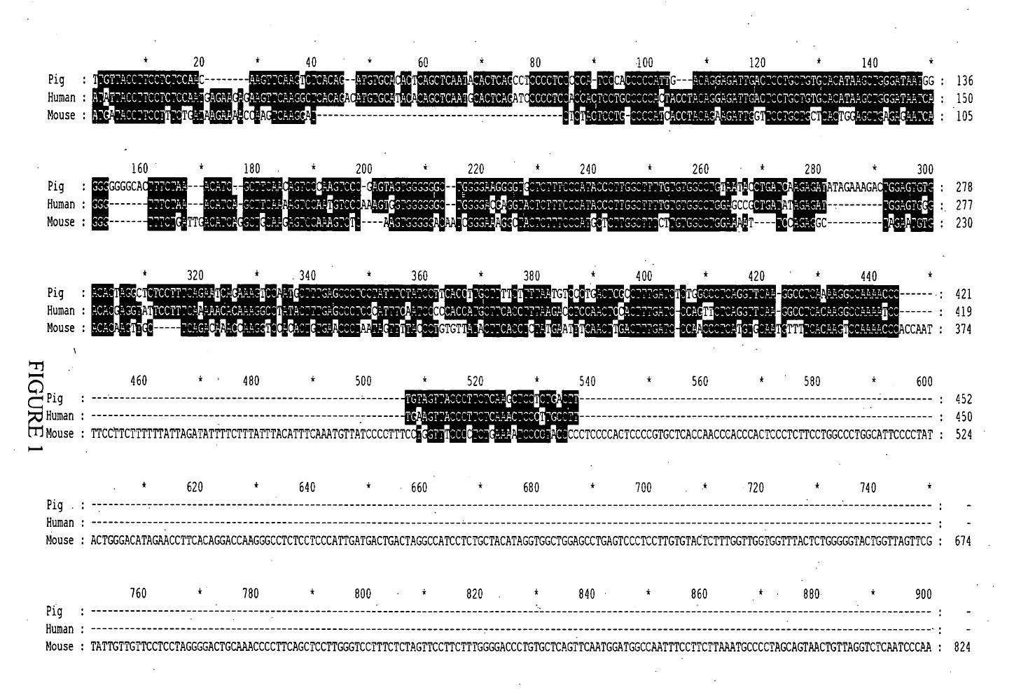

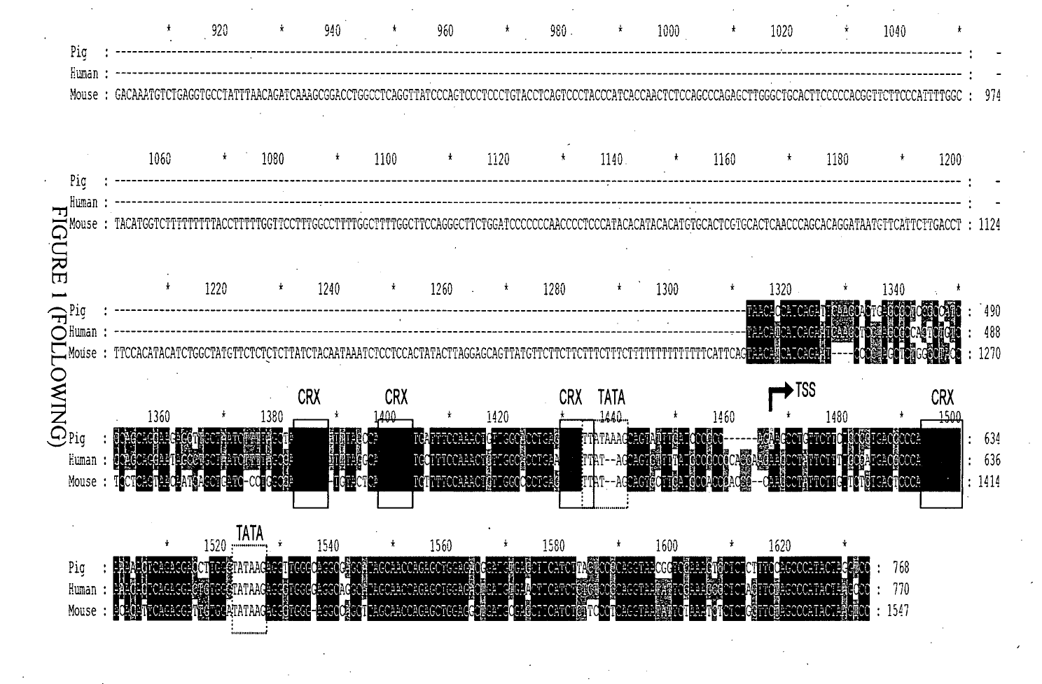

In order to determine the promoter sequence to be used to drive expression in cones, sequence alignment between mouse, human and porcine promoter region of the Cone Arrestin-3 gene (also called mouse Cone Arrestin/CAR in mouse) was performed. Alignment of the pig promoter sequence with human sequence shows more than 70% identity from base −1250 to base +123; with pig to mouse only from base −100 to base +123, from base −450 to −350 and from base −800 to −700 (FIG. 1).

Firstly a conserved region in the three species of about 250 base pairs was selected (−121, +123). Secondly, because the upstream sequence in mouse differed from the human and porcine, a longer sequence of about 720 base pairs from the porcine promoter was selected (−598, +123).

The respective sequence of the selected porcine Arrestin 3 short and long promoter regions are described in the SEQ ID NO:5 and SEQ ID NO:6.

### Example 2

### Lentiviral Constructs

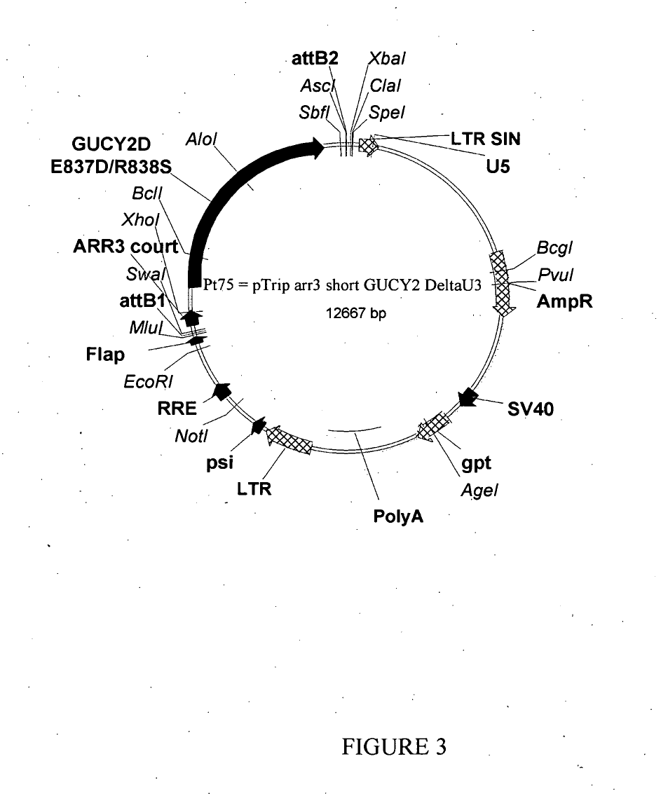

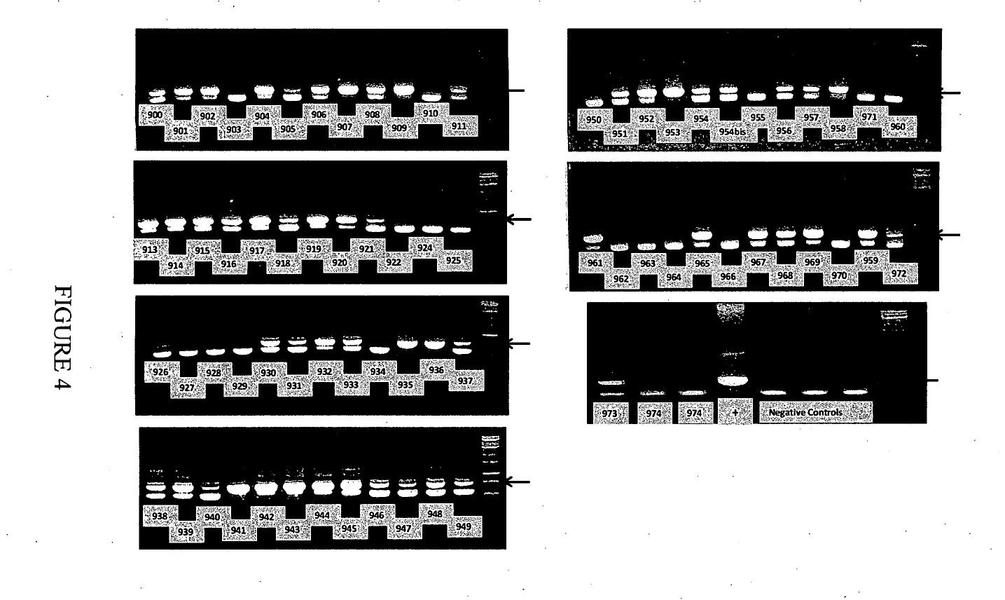

The expressing cassette composed of the short (−121, +123) or long (−598, +193) porcine Arrestin3 promoter sequence followed by the human mutant GUCY2D cDNA (bearing mutations E837D/R838S) were subcloned into the pTrip-RFA plasmid to generate respectively the plasmid called Pt75 (FIG. 3) and Pt71 (FIG. 2). Lentiviral stocks were produced by triple transfection in HEK293T cells with a transcomplementing plasmid p8.9 and a pVSV-G envelope plasmid, as previously described (Grandchamp N. et al., Genet Vaccines Ther. 2011 Jan. 4; 9(1):1). Titration of the stocks was performed using the ELISA technique to measure the capsid p24 concentration, as previously described (Piedrahita, D. et al. (2010). J. Neurosci. 30:13966-13976).

The cDNA nucleic acid sequence of the wild-type human GUCY2D gene is herein identified as SEQ ID NO:1 and the amino acid sequence of the wild-type human GUCY2D polypeptide is herein identified as SEQ ID NO:2.

The cDNA nucleic acid sequence of the human GUCY2D E837D/R838S mutant is herein identified as SEQ ID NO:3 and the amino acid sequence of the human GUCY2D E837D/R838S mutant polypeptide is herein identified as SEQ ID NO:4.

The respective sequence of the final constructs pt75 and pt71 are respectively described in the SEQ ID NO:7 and SEQ ID NO:8.

### Example 3

### Generation of Transgenic Animals

Embryos were produced from Large-White gilts that were approximately 9 months of age and weighed at least 120 kg at time of use. Super-ovulation was achieved by feeding, between day 11 and 15 following an observed oestrus, 20 mg altrenogest (Regumate, Hoechst Roussel Vet. Ltd., Milton Keynes, UK) once daily for 4 days and 20 mg altrenogest twice on the fifth day. On the sixth day, 1500 international units (IU) of eCG (PMSG, Intervet UK Ltd, Cambridge, UK) were injected at 8:00 P.M. Eighty three hours later 750 IU hCG (Chorulon, Intervet UK Ltd, Cambridge, UK) were injected.

Donors gilts were inseminated twice 6 h apart after exhibiting heat generated following super-ovulation. Recipient females were treated identically, to donor gilts but remained un-mated. Embryos were surgically recovered from mated donors by mid-line laparotomy under general anesthesia on day 1 following oestrus. (Heat=estrus Day 0). Embryos were injected with the virus constructs by sub-zonal injection into the per-vitalin space using fine glass needles under an inverted microscope.

Immediately following treatment fertilized embryos were transferred to recipient gilts following a mid-line laparotomy under general anesthesia. During surgery, the reproductive tract was exposed and embryos were transferred into the oviduct of recipients using a 3.5 French gauge tomcat catheter.

Animals investigated in this study were hemizygous male and female transgenic pigs and non-transgenic (littermate) control animals. All animal experiments were carried out following ethical review and conducted under The Animal (Scientific Procedures) Act 1986 (UK).

### Example 4

### Genotyping of Pigs and Transgene Expression in the Retina

Genomic DNA was prepared from ear clips by proteinase K digestion in lysis buffer followed by phenol/chloroform extraction. Offspring were genotyped by PCR (FIG. 4) using lentiviral backbone-specific primers designed against the sequence of the lentivirus:

(SEQ ID NO: 9)
Forward: 5′ caatttgctgagggctattgag 3′
(SEQ ID NO: 10)
Reverse: 5′ ctgtccctgtaataaacccg 3′

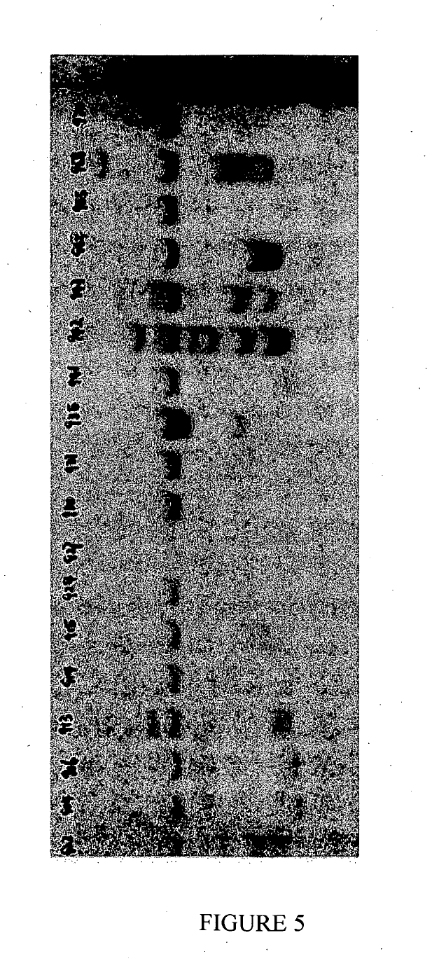

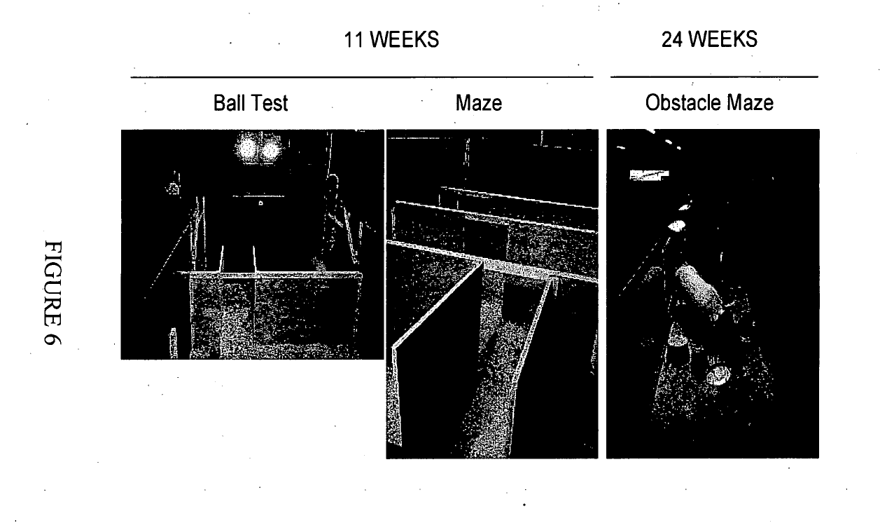

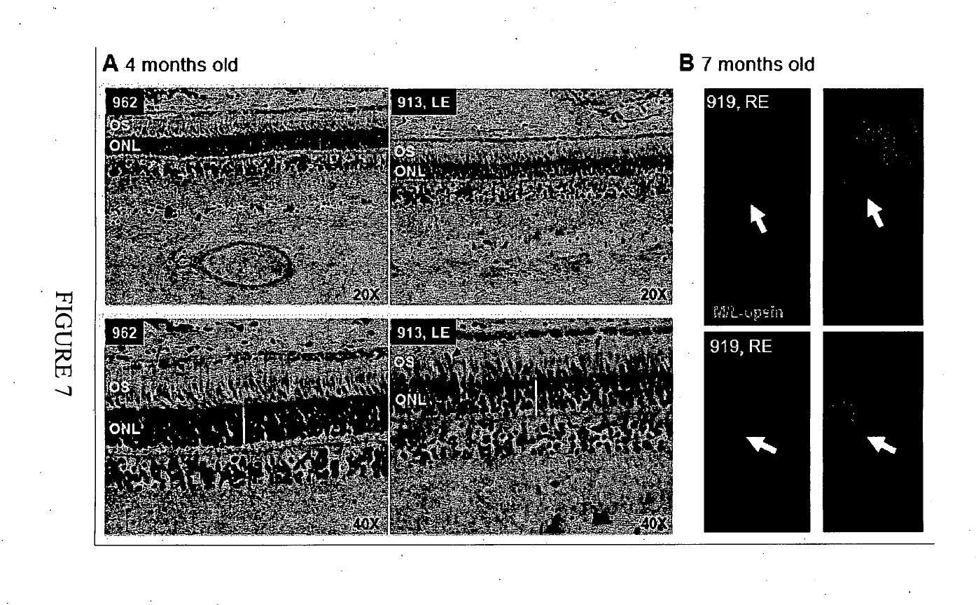

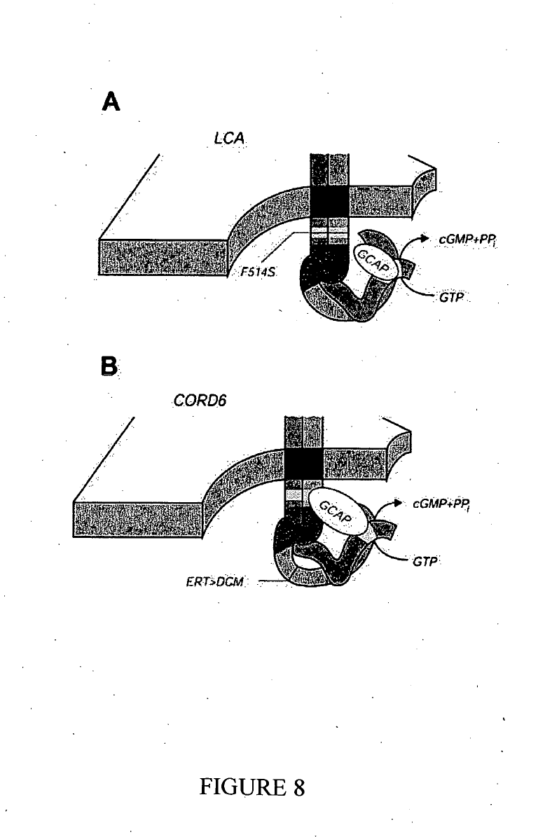

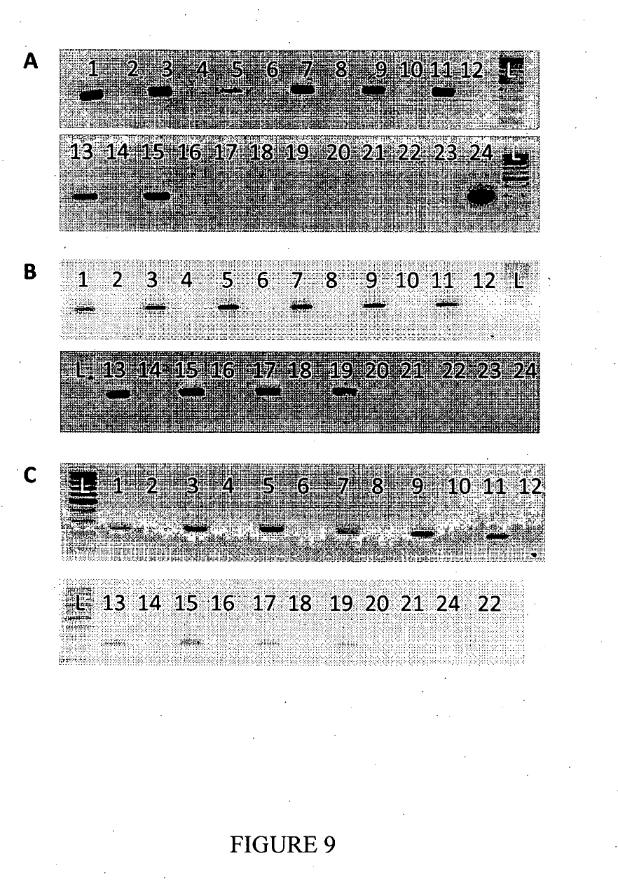

For Southern blot analysis, genomic DNA (aliquots of 20 μg) extracted as above, was digested with the restriction enzyme EcoRI and hybridized with a DIG-labeled probe directed towards the GUCY2D sequence. The southern blots allowed determining the number of vector integration within the pig genome. (FIG. 5)

SEQ ID NO:14 (sequence of a GUCY2D fragment) was used as probe and was synthesized by PCR with the following primer sites:

(SEQ ID NO: 11)
Forward sequence: 5' agatcatcctgaccgtggac 3'
(SEQ ID NO: 12)
Reverse sequence: 5' gaccacaccttcgacctgtt 3'.
(SEQ ID NO: 14)
AGATCATCCTGACCGTGGACgacatcacctttctccacccacatgggggcacctctcgaaaggtggcccagggga
gtcgatcaagtctgggtgcccgcagcatgtcagacattcgcagcggccccagccaacacttggacagccccaacattggtgtctatga
gggagacagggtttggctgaagaaattcccaggggatcagcacatagctatccgcccagcaaccaagacggccttctccaagctcca
ggagctccggcatgagaacgtggccctctacctggggcttttcctggctcggggagcagaaggccctgcggccctctgggagggca
acctggctgtggtctcagagcactgcacgcggggctctcttcaggacctcctcgctcagagagaaataaagctggactggatgttcaa
gtcctccctcctgctggaccttatcaagggaataaggtatctgcaccatcgaggcgtggctcatgggcggctgaagtcacggaactgc
atagtggatggcagattcgtactcaagatcactgaccacggccacgggagactgctggaagcacagaaggtgctaccggagcctccc
agagcggaggaccagctgtggacagccccggagctgcttagggacccagccctggagcgccggggaacgctggccggcgacgt
ctttagcttggccatcatcatgcaagaagtagtgtgccgcagtgccccttatgccatgctggagctcactcccgaggaagtggtgcaga
gggtgcggagcccccctccactgtgtcggcccttggtgtccatggaccaggcacctgtcgagtgtatcctcctgatgaagcagtgctg
ggcagagcagccggaacttcggccctccatgGACCACACCTTCGACCTGTT

RNA from retina tissue collected from pigs at 18 month of age were also prepared. Tissue samples stored at −80° C. were defrosted on ice, the retina was separated from surrounding tissue and placed in 4 ml Trizol and homogenized by physical disruption using a 10 ml syringe and 3 different sized needles (in order of usage 18 G, 21 G and 25 G). The tissue was considered homogenized when the totality of the sample was passed 3 times through the smallest needle (25 G). RNA extraction was then performed according to manufacturer instructions. In column DNase digestion was performed using RNeasy Mini Kit from Qiagen, according to manufacturer instructions. Reverse transcription was performed and cDNA were amplified with specific primers for pig GAPDH cDNA (control endogenous gene), pig GUCY2D cDNA (endogenous gene) and for human mutant GUCY2D cDNA (the transgene):

Pig GAPDH forward sequence:
(SEQ ID NO: 19)
5′ GATGGTGAAGGTCGGAGTGA 3′
Pig GAPDH reverse sequence:
(SEQ ID NO: 20)
5′ AGGCATTGCTGACGATCTTG 3′
Pig GUCY 2D forward sequence:
(SEQ ID NO: 21)
5′ GAGGACCTGATCGGGGAGC 3′
Pig GUCY 2D reverse sequence:
(SEQ ID NO: 22)
5′ CACCTTGTAGACATCATGGGAG 3′
Human mutant GUCY 2D forward sequence:
(SEQ ID NO: 23)
5′ GAGGATCTGATCCGGGACA 3′
Human mutant GUCY 2D reverse sequence:
(SEQ ID NO: 24)
5′ TCTCCACCTTGTAGACATCG 3′

RT PCR results (FIG. 9) are consistent with results obtained via genotyping by PCR and Southern blot: animals that were identified as containing the transgene also express said transgene while animals in which the transgene was not detected show no transgene expression by RT−PCR. These results demonstrate that the transgenic animals do express the human dominant negative Gucy2D allele in the retina, responsible for the observed phenotype.

### Example 5

### Non-Invasive Phenotyping of Pigs by Electroretinograms (ERG) and Optical Coherence Tomography (OCT)

Retinal function was measured by electroretinogram (ERG) using the ISCEV procedure. In photopic conditions (cone activity measurement), single stimuli of 3 candela steradian per square meter (cds/m²) and 10 cds/m² as well as repeated flashes (flicker) of 10 to 30 Hz at 3 cds/m² were used. In photopic conditions, measures were recorded at 11 weeks age (Table 1) and 24 weeks age (Table 2) and 52 weeks of age (not shown).

The retinal response in scotopic conditions (rod activity measurement) was also performed on some animals at the age of 24 weeks. In this case, single flashes of 0.01, 3 and 10 cds/m² after dark adaptation for at least 20 minutes were recorded.

For each response, the amplitude and the a-wave and b-wave lengths were quantified (Tables 1 and 2 for respectively 11 and 24 weeks of age measurements, not shown for 52 weeks of age measurements). Control animals (non transgenic littermates) were also recorded in the same conditions. For each time point considered, results show dramatic reduction of electrical activity in representative transgenic animals as compared to age-matched control animals, revealing that the transgenesis has induced severe alteration in the retina activity.

TABLE 1
Phenotyping of pigs retinal activity by ERG at 11 weeks of age

| | | | | | ERG 11 weeks | | | | | | | | | | | | |
|---|---|---|---|---|---|---|---|---|---|---|---|---|---|---|---|---|---|
| | | | | trans- | | photopic | | | | | | | | | | | |
| | | | | gene | | | | | | 10 HZ | | 30 HZ | | | | | |
| identi- | gen- | eye | geno- | copy | | unique 3 cds/m2 | | | | 3 cds/m2 | | 3 cds/m2 | | unique 10 cds/m2 | | | |
| fication | der | colour | type | number | eye | a-lat | b-lat | a-ampl | b-ampl | b-lat | b-ampl | b-lat | b-ampl | a-lat | b-lat | a-ampl | b-ampl |
| 904 | M | | LONG | 1 | L | 13 | 28 | 7.11 | 69 | 28 | 74.7 | 25 | 67.2 | 14 | 28 | 8.84 | 85 |
| 906 | M | | LONG | 1 | R | 11 | 26 | 13 | 109 | 26 | 233 | 23 | 203 | 12 | 24 | 33.1 | 278 |
| 907 | M | | LONG | 5 | L | 12 | 29 | 9.81 | 107 | 28 | 118 | 25 | 118 | 13 | 34 | 24.1 | 143 |
| 908 | M | | LONG | 2 | | 12 | 26 | 21 | 129 | 26 | 163 | 24 | 183 | 12 | 26 | 29.8 | 193 |
| 909 | F | | LONG | 3 | R | 12 | 25 | 19.3 | 56.1 | 26 | 61.9 | 24 | 62.4 | 12 | 29 | 17.6 | 94.9 |
| 913 | M | | LONG | 5 | L | 12 | 24 | 0.633 | 25 | 24 | 17.1 | 24 | 20.2 | 10 | 26 | 14.3 | 40.7 |
| | | | | | R | 12 | 27 | 15.8 | 80.9 | 26 | 123 | 24 | 110 | 13 | 30 | 18.3 | 176 |
| 914 | M | | LONG | 2 | L | 11 | 25 | 7.47 | 126 | 26 | 127 | 24 | 84.5 | 11 | 25 | 18.4 | 134 |
| 915 | M | | LONG | 2 | R | 11 | 25 | 15.4 | 127 | 25 | 127 | 22 | 89.3 | 12 | 26 | 19.5 | 164 |
| 917 | M | bleu | LONG | 3 | L | 13 | 28 | 11.4 | 13.2 | 28 | 25.3 | 24 | 19.2 | 13 | 34 | 3.69 | 39.3 |
| | | bleu | | | R | 14 | 31 | 0.581 | 5.8 | 28 | 11.7 | 24 | 7.69 | 14 | 34 | 3.21 | 16.6 |
| 918 | M | | LONG | 3 | R | 14 | 29 | 5.08 | 30.3 | 28 | 33.2 | 25 | 18.1 | 13 | 32 | 2.96 | 20 |
| | | | | | L | 12 | 30 | 2.39 | 53.2 | 29 | 31.1 | 24 | 34.4 | 14 | 32 | 13.8 | 87.7 |
| 919 | M | | LONG | 6 | R | | | | | | | | | 13 | 28 | 9.49 | 28.6 |
| | | clair | | | L | 11 | 26 | 14 | 74.9 | 28 | 116 | 24 | 84.7 | 13 | 33 | 10.7 | 154 |
| 920 | M | | LONG | 4 | L | 12 | 27 | 21.8 | 122 | 27 | 126 | 24 | 125 | 12 | 29 | 12.3 | 105 |
| 924 | M | | neg | 0 | R | 11 | 25 | 10.4 | 190 | 25 | 81.8 | 24 | 94.3 | 12 | 29 | 12 | 87.6 |
| 927 | F | | neg | 0 | R | 13 | 26 | 32.6 | 160 | 27 | 213 | 24 | 184 | 12 | 28 | 36.8 | 276 |
| | | | | | L | 10 | 26 | 6 | 105 | 26 | 80.5 | 24 | 103 | 12 | 28 | 36.5 | 133 |
| 928 | F | | neg | 0 | R | 14 | 28 | 15.4 | 118 | 27 | 97.6 | 24 | 96.2 | 13 | 30 | 33.9 | 198 |
| 929 | F | | neg | 0 | L | 12 | 28 | 23.2 | 162 | 27 | 193 | 24 | 166 | 11 | 32 | 19.4 | 338 |
| 970 | F | | neg | 0 | L | 12 | 26 | 16 | 144 | 26 | 211 | 24 | 201 | 12 | 30 | 32.6 | 297 |
| 974 | F | | neg | 0 | R | 11 | 25 | 35 | 225 | 25 | 246 | 24 | 246 | 12 | 28 | 66.8 | 313 |

| | unique 3 cds/m2 | | | | 10 HZ 3 cds/m2 | | 30 HZ 3 cds/m2 | | unique 10 cds/m2 | | | |
|---|---|---|---|---|---|---|---|---|---|---|---|---|
| | a-w lat | b-w lat | a-w am [symbol] | b-w am [symbol] | b-w lat | b-w am [symbol] | b-w lat | b-w am [symbol] | a-w lat | b-w lat | a-w am [symbol] | b-w am [symbol] |
| mean non-transgenic | 11.9 | 26.3 | 19.8 | 157.7 | 26.1 | 160.4 | 24.0 | 155.8 | 12.0 | 29.3 | 34.0 | 234.7 |
| SEM non-transgenic | 0.5 | 0.5 | 4.5 | 16.8 | 0.4 | 29.0 | 0.0 | 24.3 | 0.2 | 0.6 | 7.0 | 39.3 |
| Mean selected | | | 3.9 | 40.9 | | 36.4 | | 32.7 | | | 8.8 | 51.6 |
| transgenic | | | | | | | | | | | | |
| SEM selected | | | 1.4 | 9.8 | | 9.5 | | 9.5 | | | 1.6 | 12.2 |
| transgenic | | | | | | | | | | | | |
| T-test, p | | | 0.00566 | 0.00002 | | 0.00088 | | 0.00026 | | | 0.00084 | 0.00021 |

[symbol]

indicates data missing or illegible when filed

TABLE 2
Phenotyping of pigs retinal activity by ERG at 24 weeks of age

| | ERG 24 weeks | | | | | | | | | | | | |
|---|---|---|---|---|---|---|---|---|---|---|---|---|---|
| | | photopic | | | | | | | | | | | |
| | | unique 3 cds/m2 | | | | 10 HZ | | 30 HZ | | unique 10 cds/m2 | | | |
| identification | eye | a-lat | b-la [symbol] | a-ampl | b-ampl | b-lat | b-amp [symbol] | b-lat | b-amp [symbol] | a-lat | b-lat | a-ampl | b-ampl |
| 904 | L | 12 | 25 | 5 | 57 | 25 | 106 | 22 | 80 | 12 | 30 | 12 | 89 |
| 906 | R | 11 | 26 | 28 | 212 | 25 | 248 | 23 | 296 | 11 | 30 | 55 | 485 |
| 907 | L | | | | | | | | | | | | |
| 908 | L | 13 | 27 | 7 | 86 | 25 | 78 | 24 | 62 | 14 | 31 | 41 | 141 |
| 909 | R | 12 | 27 | 7 | 52 | 26 | 60 | 23 | 39.4 | 11 | 32 | 5 | 46 |
| 913 | L | | | | | | | | | | | | |
| | R | | | | | | | | | | | | |
| 914 | L | 12 | 25 | 4 | 36 | 26 | 305 | 24 | 232 | 12 | 31 | 53 | 318 |
| 915 | R | 13 | 28 | 33 | 240 | 29 | 120 | 26 | 73 | 13 | 32 | 32 | 283 |
| 917 | L | 12 | 29 | 7 | 28 | 28 | 33 | 22 | 20 | 11 | 33 | 15 | 80 |
| | R | 12 | 29 | 23 | 123 | 28 | 164 | 22 | 128 | 12 | 31 | 30 | 218 |
| 918 | R | 10 | 31 | 2 | 26 | 28 | 20 | 25 | 16 | 11 | 35 | 13 | 35 |
| | L | 11 | 29 | 4 | 98 | 28 | 138 | 25 | 127 | 14 | 27 | 44 | 156 |
| 919 | R | 12 | 28 | 2 | 6 | 28 | 6 | 22 | 6 | 11 | 32 | 6 | 12 |
| | L | | | | | | | | | | | | |
| 920 | L | 12 | 26 | 6 | 58 | 26 | 77 | 24 | 55 | 12 | 32 | 3 | 72 |
| 924 | R | 12 | 27 | 26 | 139 | 26 | 150 | 24 | 130 | 12 | 30 | 22 | 196 |
| 927 | R | 12 | 25 | 16.2 | 86.2 | 24 | 70.3 | 22 | 67.8 | 11 | 27 | 6.03 | 104 |
| | L | 12 | 26 | 15 | 132 | 25 | 117 | 23 | 98 | 11 | 28 | 17 | 134 |
| 928 | R | 12 | 26 | 18.4 | 108 | 26 | 121 | 23 | 101 | 12 | 31 | 20.7 | 139 |
| 929 | L | 11 | 25 | 13 | 97 | 25 | 121 | 23 | 83 | 11 | 28 | 8 | 72 |
| 970 | L | 12 | 25 | 26 | 172 | 25 | 228 | 23 | 203 | 12 | 28 | 48 | 307 |
| 974 | R | 12 | 26 | 13 | 94 | 26 | 102 | 24 | 79 | 12 | 30 | 22 | 117 |

| ERG 24 weeks | | |
|---|---|---|
| scotopic | | |
| 0.01 cds/m2 | unique 3 cds/m2 | unique 10 cds/m2 |

| identification | b-lat | b-ampl | a-lat | b-lat | a-ampl | b-ampl | a-lat | b-la [symbol] | a-ampl | b-ampl |
|---|---|---|---|---|---|---|---|---|---|---|
| 904 | 76 | 38 | 13 | 39 | 21 | 225 | 12 | 39 | 71 | 433 |
| 906 | | | | | | | | | | |
| 907 | | | | | | | | | | |
| 908 | | | | | | | | | | |
| 909 | 71 | 22.1 | 18 | 39 | 4 | 72.8 | 18 | 37 | 15.9 | 43.6 |
| 913 | | | | | | | | | | |
| 914 | | | | | | | | | | |
| 915 | | | | | | | | | | |
| 917 | 77 | 29 | 12 | 37 | 10 | 27 | 13 | 31 | 14 | 28 |
| | 57 | 29 | | | | | 11 | 42 | 7 | 11 |
| 918 | 73 | 27 | 12 | 42 | 6 | 18 | 14 | 38 | 10 | 12 |
| 919 | 0 | 0 | 12 | 38 | 21 | 21 | 13 | 40 | 3 | 13 |
| 920 | | | | | | | | | | |
| 924 | 80 | 26 | 14 | 34 | 37 | 304 | 14 | 34 | 59 | 346 |
| 927 | 67 | 38.1 | 12 | 36 | 29.9 | 81.8 | 19 | 33 | 34.4 | 112 |
| | 67 | 63 | 11 | 34 | 48.8 | 311 | 11 | 32 | 117 | 471 |
| 928 | | | | | | | | | | |
| 929 | 80 | 34 | 11 | 38 | 16 | 91 | 12 | 33 | 60 | 214 |
| 970 | 92 | 14 | 12 | 31 | 20 | 134 | 18 | 32 | 33 | 99 |
| 974 | 86 | 10 | 13 | 35 | 14 | 78 | 15 | 35 | 1 | 77 |

| | unique 3 cds/m2 | | | | 10 HZ | | 30 HZ | | unique 10 cds/m2 | | |
|---|---|---|---|---|---|---|---|---|---|---|---|
| | a-w lat | b-w lat | a-w am [symbol] | b-w am [symbol] | b-w lat | b-w am [symbol] | b-w lat | b-w am [symbol] | a-w lat | b-w lat | a-w am [symbol] |
| [symbol] an non- | 11.9 | 25.7 | 18.2 | 118.3 | 25.3 | 129.9 | 23.1 | 108.8 | 11.6 | 28.9 | 20.5 |
| transge [symbol] | | | | | | | | | | | |
| [symbol] M non- | 0.2 | 0.3 | 2.3 | 12.6 | 0.3 | 20.2 | 0.3 | 18.8 | 0.2 | 0.6 | 5.6 |
| transge [symbol] | | | | | | | | | | | |
| Mean | | | 10.78 | 56.50 | | 88.75 | | 68.86 | | | 19.33 |
| selected | | | | | | | | | | | |
| SEM | | | 3.40 | 18.65 | | 42.06 | | 21.79 | | | 6.57 |
| selected | | | | | | | | | | | |
| T-test, p | | | 0.00001 | 0.00007 | | 0.00450 | | 0.00515 | | | 0.09066 |

| | unique | | | | | | | | | | |
|---|---|---|---|---|---|---|---|---|---|---|---|
| | 10 cds/m2 | 0.01 cds/m2 | | unique 3 cds/m2 | | | | unique 10 cds/m2 | | | |
| | b-w am [symbol] | b-w lat | b-w am [symbol] | a-w lat | b-w lat | a-w am [symbol] | b-w am [symbol] | a-w lat | b-w lat | a-w am [symbol] | b-w am [symbol] |
| [symbol] an non- | 152.7 | 78.7 | 30.9 | 12.2 | 34.7 | 27.6 | 166.6 | 14.8 | 33.2 | 50.7 | 219.8 |
| transge [symbol] | | | | | | | | | | | |
| [symbol] M non- | 31.8 | 4.1 | 7.8 | 0.5 | 1.0 | 5.5 | 45.3 | 1.3 | 0.5 | 15.9 | 64.7 |
| transge [symbol] | | | | | | | | | | | |
| Mean | 122.25 | | nd | | | 8.00 | 22.50 | | | 8.50 | 16.00 |
| selected | | | | | | | | | | | |
| SEM | 46.79 | | | | | 2.00 | 3.67 | | | 2.33 | 4.02 |
| selected | | | | | | | | | | | |
| T-test, p | 0.02329 | | | | | 0.03766 | 0.05097 | | | 0.04703 | 0.02201 |

[symbol]

indicates data missing or illegible when filed

Retina morphology of some transgenic and control animals was also analysed using OCT (Optical Coherence Tomography) at the age of 24 months and 52 months. OCT is a non-invasive technology used for imaging the retina, the multi-layered sensory tissue lining the back of the eye. OCT which allows seeing cross-sectional images of the retina is revolutionizing the early detection and treatment of eye conditions such as macular holes, pre-retinal membranes, macular swelling and even optic nerve damage. OCT uses the optical backscattering of light to rapidly scan the eye and describe a pixel representation of the anatomic layers within the retina. Each of these ten important layers can be differentiated and their thickness can be measured.

### Example 6

### Visual Behavior Test

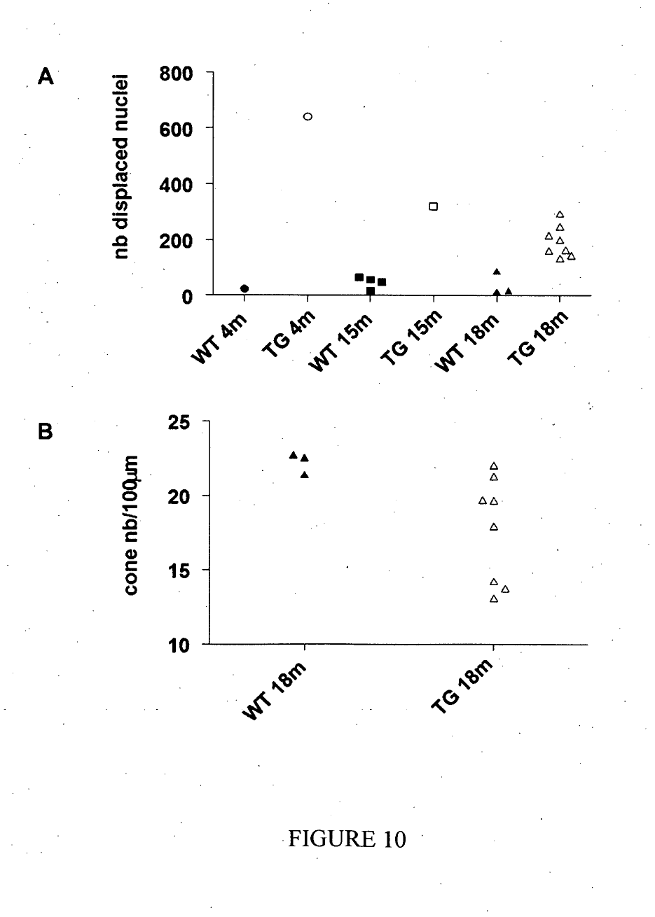

At the age of 11 weeks, visual behavior of animals was evaluated by two tests (FIG. 6). The first one, the “ball test” consists in moving vertically a ball at the left or right side of the pigs' head, which head is restrained by two boards. The pig reaction is video recorded and classified as “surprise” (a jump with surprise is scored ++) to indifference (scored −).

The second test consists of a maze that was constructed in a corridor flanked by two boards. A large colored ball is visible from the maze start. The time necessary for the animals to reach the ball and pass both “doors” is measured.

At 24 weeks age, in condition of strong light (1000 to 2400 lux), a maze was constructed which consisted of a corridor where various colored obstacles (circulation cones, buckets and a suspended flying-disc) were placed at fix points (FIG. 6). The time necessary for the animals to pass the maze was measured. Animals were also video recorded during this test to analyze their behavior (obstacle sniffing, licking, etc.). A full score was given to animals that pass the obstacles by walking around and starring at them. Contribution of senses (sight, and non-sight senses such as olfaction and tasting) were scored. Each contribution (visual or other) was scored between 0 (no contribution) to 1 (sense used for each obstacle) for the whole maze (5 obstacles in total). A final score was calculated that determines the relative contribution of sight as compared to other senses (visual versus others) (Table 3).

A similar experiment was reproduced with animals aged of 52 weeks and animal performance was evaluated as described above (not shown).

For each time point considered, behavioral evaluation of the animals show a dramatic reduction of visual function in representative transgenic animals as compared to age-matched control animals, revealing that the transgenesis has induced severe alteration of the visual function of the animals.

TABLE 3
Visual behavior tests (“Ball” test, Maze test and Obstacle maze test).
A picture of the tests settings is shown in FIG. 6.

| | | | | | | | | test 24 weeks | | | |
|---|---|---|---|---|---|---|---|---|---|---|---|
| | | | | | test 11 weeks | | | Obstacles | | | |
| | | | | | Ball test | | | | contribution of | contribution os | |
| | | | | | | amplitude | maze | | vision 1 = use | other senses 1 = always | score |
| | | eye | | copy | | of | best | time | vision 0 = do | sniff, touch or taste | vision − other |
| identification | gender | colour | genotype | number | eye | reaction | time (s) | (s) | not use vision | 0 = never | senses = |
| 902 | M | | LONG | 3 | L | | 6 | 22 | 0.75 | 0.29 | 0.46 |
| 904 | M | | LONG | 1 | L | | 3 | 37 | 0.75 | 0.57 | 0.18 |
| 906 | M | | LONG | 1 | R | | 6 | 17 | 1.00 | 0.71 | 0.29 |
| 907 | M | | LONG | 5 | L | | 10 | 25 | 1.00 | 0.14 | 0.86 |
| 908 | M | | LONG | 2 | | | 6 | 27 | 1.00 | 0.43 | 0.57 |
| 909 | F | | LONG | 3 | R | | 15 | 38 | 1.00 | 0.71 | 0.29 |
| 913 | M | | LONG | 5 | L | + | 3 | | | | |
| | | | | | R | + | | | | | |
| 914 | M | | LONG | 2 | L | ++ | 4 | 25 | 1.00 | 0.00 | 1.00 |
| 915 | M | | LONG | 2 | R | | 2 | 18 | 1.00 | 0.00 | 1.00 |
| 917 | M | bleu | LONG | 3 | L | ++ | 7 | 50 | | | 0.00 |
| | | bleu | | | R | ++ | | | 1.00 | 1.00 | |
| 918 | M | | LONG | 3 | R | ++ | 4 | 13 | | | 0.86 |
| | | | | | L | | | | 1.00 | 0.14 | |
| 919 | M | | LONG | 6 | R | + | 5 | 47 | | | −0.75 |
| | | clair | | | L | + | | | 0.25 | 1.00 | |
| 920 | M | | LONG | 4 | L | | 3 | 67 | 1.00 | 0.57 | 0.43 |
| 924 | M | | neg | | R | | 4 | | | | |
| 927 | F | | neg | | R | | 2 | 28 | 1.00 | 0.14 | 0.86 |
| 928 | F | | neg | | R | ++ | 5 | 10 | 1.00 | 0.29 | 0.71 |
| 929 | F | | neg | | L | | 6 | 16 | 1.00 | 0.43 | 0.57 |
| 970 | F | | neg | | L | | | 9 | 1.00 | 0.00 | 1.00 |
| 974 | F | | neg | | R | ++ | 10 | 17 | 1.00 | 0.00 | 1.00 |
| 964 | F | | neg | | R | ++ | | 6 | 1.00 | 0.00 | 1.00 |

### Example 7

### Histology

Two transgenic animals and age-matched controls were killed at the age of 4 and 7 months and eyes removed for histological studies (FIG. 7). Frozen eyes were sectioned by cryostat and microtome. Eyes were oriented and sections close to the optic nerve were obtained and compared to an age-matched control eye.

Paraffin sections were stained by hematoxy-eosin (FIG. 7A) while cryostat sections were analysed by immunolabeling directed against cone cells using the M/L-opsin antibody and PNA as described in Bemelmans et al. 2006, PLoS Med Oct; 3(10):e347 (FIG. 7B). At 4 months, the left eye of pig 913 shows displaced photoreceptor nuclei in the layer of the outer segments (arrows). Moreover a clear decrease in the density of photoreceptor nuclei appears compared to a control eye (see also the white bars which have the same size). At 7 months of age the pig 919 also has mislocalised nuclei in the outer segment layer (arrows).

At 18 months of age, nine transgenic animals were sacrificed. While the right eyes were used for RNA extraction (see example 4), the left eyes were processed for histology as described above. Quantifications were performed on these sections (one section per animal) to assess for the number of displaced nuclei (for the entire section) and the number of cones labeled by PNA (in the central region, 1.6 mm superior to the optic nerve head), results are shown on FIG. 10. Transgenic animals have an increased number of displaced nuclei as compared to non-transgenic controls (FIG. 10A). Moreover some transgenic animals have a reduced number of cones as assessed by PNA labeling (FIG. 10B). PNA labeling reflects the integrity of the extracellular matrix surrounding cone inner segments. Its decrease indicates a reduction of the cone number in the transgenic animals as compared to age-matched control animals, thus confirming that transgenesis leads to retinal abnormality and affected cones.

### Example 8

### Transgenic Mice Bearing the GUCY2D Dominant Allele do not Show the Same Phenotype as Pigs

Mouse embryos were injected with a similar lentiviral vector carrying a mouse cone arrestin promoter controlling the expression of the green fluorescent protein (GFP) gene. The GFP expression was strong in cone cells, and some low expression of the GFP was present in some columns of rods, indicating that the construct specifically drive high expression of the transgene in cone cells.

Another group of mice was injected with the pig long Arrestin3-GFP-II. The fluorescence was detected in the retina using eye fundus imaging. Finally, other mice received the long Arrestin3-GUCY2D mutated transgene (using Pt71 of SEQ ID NO:8). These animals show no reduction of retinal activity in photopic condition during the adulthood (first 6 months of life), although these animals displayed expression of the transgene as detected by RT−PCR in the retina. Consistently, no obvious retinal structure changes were observed.

These results show that the transgenic mice carrying the lentiviral vector corresponding to Pt71 do not display a cone dystrophy phenotype at the age of 6 months.

These results further show that transgenic pigs present more rapidly a decrease and an alteration of the cone function than mice receiving the same construct highlighting the advantage to work with the pig model, which shows a pattern of cone loss function closer to the human disease.

1-20. (canceled)

21. A transgenic pig as a model for studying a cone affecting disease, in particular a cone- or cone-rod-dystrophy, comprising a recombinant nucleic acid, stably integrated in its genome, encoding a dominant negative human guanylate-cyclase-2D (GUCY2D) protein, the recombinant nucleic acid being operably linked to a promoter active in retinal cone cells.

22. The transgenic pig according to claim 21, wherein the dominant negative human GUCY2D protein comprises at least one mutation, in the region located between residues 816 and 861 of SEQ ID NO: 2, which is responsible for the appearance of a CORD6 cone dystrophy in a human being.

23. The transgenic pig according to claim 22, wherein the mutation is a non conservative substitution of at least one residue selected from residue 837, 838 and 839 of SEQ ID NO: 2.

24. The transgenic pig according to claim 22, wherein the recombinant nucleic acid encodes the GUCY2D protein of SEQ ID NO: 4 comprising the E837D and the R838S mutations.

25. The transgenic pig according to claim 21, wherein the promoter active in retinal cone cells is selected from the short cone Arrestine promoter of SEQ ID NO: 5, the long cone Arrestine promoter of SEQ ID NO: 6 and any functional variant thereof.

26. A genetically modified cell derived from a transgenic pig according to claim 21.

27. The cell according to claim 26, wherein said cell is selected from a stem cell, in particular an induced pluripotent stem cell (iPS cell), a germ cell, a gamete and a somatic cell.

28. Nucleus of a cell according to claim 26.

29. A population of cells derived from a cell according to claim 26.

30. A fertilized egg derived from the transgenic pig model as defined in claim 21.

31. A method for evaluating the efficacy of a compound for preventing or treating a cone affecting disease, said method comprising the steps of i) providing the pig model according to claim 21, ii) administering to said pig model a compound the efficacy of which is to be evaluated, and iii) evaluating the effect, if any, of the compound on the phenotype induced by the dominant negative GUCY2D protein expressed in the pig model.

32. The method of claim 31, wherein the compound is selected from a therapeutic vector, a nucleic acid, a cell, a population of cells, a drug, a functional food and a mixture thereof.

33. A method for evaluating the efficacy of an artificial retina or of a biocompatible polymer capsule, said method comprising the steps of i) providing the pig model according to claim 21, ii) grafting to said pig model an artificial retina or a biocompatible polymer capsule the efficacy of which is to be evaluated, and iii) evaluating the effect, if any, of the artificial retina or of the biocompatible polymer capsule on the phenotype induced by the mutated GUCY2D protein expressed in the pig model.

34. A process for producing a transgenic pig as a model for studying a cone affecting disease comprising the steps of:
a) providing a nucleic acid expression cassette comprising a promoter active in retinal cone cells operably linked to a recombinant nucleic acid encoding a dominant negative human guanylate-cyclase-2D (GUCY2D) protein or polypeptide,
b) placing said cassette within an embryo of a female pig under conditions in which said cassette is stably integrated into the genome of said pig; and
c) causing said embryo to go to term so as to generate a transgenic pig which is a model for studying a cone affecting disease.

35. The process of claim 34, wherein the nucleic acid expression cassette is contained in a lentiviral vector produced with a plasmid containing said expression cassette, preferably with a plasmid of SEQ ID NO: 7 or 8.
